(19) United States
(12) Patent Application Publication
(10) Pub. No.: US 2022/0245270 A1
(43) Pub. Date: Aug. 4, 2022

(54) Personal Health Record System and Method using Patient Right of Access

(71) Applicant: Medblob Inc., Boston, MA (US)

(72) Inventors: Pablo Maceiras De Araujo, Montevideo (UY); Peter Ierardi, Amherst, NH (US); Richard S. Tannenbaum, New York, NY (US)

(21) Appl. No.: 17/649,579

(22) Filed: Feb. 1, 2022

Related U.S. Application Data
(60) Provisional application No. 63/144,192, filed on Feb. 1, 2021.

(51) Int. Cl. G06F 21/62; H04L 9/40; G16H 10/60

(52) U.S. Cl. CPC G06F 21/6218; H04L 2463/082; G16H 10/60; H04L 63/0838

(57) ABSTRACT

Methods and systems for managing access to patient healthcare data, using a patient right of access called for in relevant industry law, simplifies processes of digitally identifying, granting, querying, extracting, and transforming a personal health record (PHR). An identity of the patient, an execution of a right of access form by the patient, and presence of the right of access form within memory of the system, are verified by the system. A query for healthcare data pertaining to the patient may be relayed by the system from a querying entity to a possessing entity in possession of the requested healthcare data. The requested data may be received by the system and stored within a database associated with the patient, creating or augmenting a PHR for the patient. The requested data may be anonymized by the system for a researcher studying a pool of patients.

### RELATED APPLICATION

This application claims the benefit of U.S. Provisional Application No. 63/144,192, filed on Feb. 1, 2021. The entire teachings of the above application are incorporated herein by reference.

### BACKGROUND

Under the provisions of the Health Insurance Portability and Accountability Act of 1996 (HIPAA), the Health Information Technology for Economic and Clinical Health Act of 2009 (HITECH), and the Federal Trade Commission (FTC), health information, with few exceptions, can only be shared with express permission, advance consent, and authorization of the patient. Thus, patients, providers, payers, and researchers (PPPRs) have struggled to access clinical data for purposes of maintaining a personal health record (PHR), administering treatment, analyzing insurance claims, and aggregating patient data for clinical study respectively.

Current electronic medical records (EMR) systems, PHRs, and health information exchanges (HIEs) suffer from legal restrictions in storing and transmitting health information, and from liabilities associated with improper disclosures of protected healthcare information. PPPRs therefore face restrictions that limit cloud-based electronic data exchange and storage of HIPAA and HITECH regulated health information, contributing to increases in preventable patient injuries and deaths, increases in the cost of medical insurance, and reductions in speed of innovation in various medical areas.

### SUMMARY

Current methods for extraction, transformation, and loading (ETL) of patient information rely heavily on manual processes of clinical staff and patients, who must interact with a system of record in order to manage and update medical information. Other electronic query systems require a provider's authorization and are only able to send structured and unstructured data between facilities that have sophisticated and/or identical versions of EMR software. This data query method does not produce the proper consent from the patient for the medical record, other than for treatment purposes, therefore limiting usefulness of the medical data for other purposes.

The present disclosure relates to a cloud-based PHR and patient relationship manager (PRM) system. The claimed methods and systems simplify processes of digitally identifying, granting, querying, extracting, and transforming a PHR, which would otherwise cost a patient a significant amount of time or money to hire a third-party PHR service.

In an example embodiment, a method of managing access to patient healthcare data includes configuring a processor to register a patient for access to an HIE system. The processor is further configured to provide login credentials to the patient. The processor is further configured to grant the patient access to the HIE system on an identity server by validating login credentials submitted by the patient against the provided login credentials. The processor is further configured to end the method or to allow the patient another login attempt if the login attempt fails. The processor is further configured to obtain, from the patient, information pertaining to the patient including an identity of the patient.

To continue with respect to the aforementioned example embodiment, the processor is further configured to validate the received identity of the patient. In some embodiments, the identity of the patient is obtained via one or more identity documents. In these embodiments, the processor is configured to validate the obtained identity documents by interfacing with an external API. Such an external API may be hosted in an official capacity by an office with a recognized authority to maintain records identifying the patient, such as by a government office. In these embodiments, the processor is configured to interface with the API via a secure connection to affirm information presented within the obtained identity documents. Such affirming may include determining if the information is true and up to date. Such interfacing may include transferring of information, wherein the information is encoded for increased security, in order to protect the patient's privacy. In the example embodiment, the processor is further configured to end the method if the patient identity is found to be invalid.

To continue, in the example embodiment, the processor is further configured to generate a right of access form and provide the right of access form to the patient. The processor is further configured to obtain an acceptance of the right of access form from the patient. Such acceptance may be represented by a signed or otherwise executed instance of the right of access form, or may be represented via an electronic signature or other electronic indication of agreement. The processor is further configured to store the right of access form in a memory device of at least one of the identity server and a user health database server. A right of access form may be understood as a document allowing an individual, who is a subject of protected health information (PHI), to invoke rights granted in 45 C.F.R. § 164.524, Access of individuals to protected health information. The right of access form may be a digital document. The processor is further configured to receive a query for healthcare data pertaining to the patient from a querying entity. A query for healthcare data may herein be referred to interchangeably as a request for healthcare data. The processor is further configured to relay the query for healthcare data toward a possessing entity. The processor is further configured to obtain the healthcare data, or an indication of an error, from the possessing entity through the HIE system. The processor is further configured to relay the obtained healthcare data, or the indication of an error, to the querying entity. The processor is further configured to create or augment a PHR by storing the obtained healthcare data in a memory device of at least one of the identity server and the user health database server.

In some embodiments, a processor may be configured to establish, on the identity server, an account profile for the patient. The processor may be further configured to populate the account profile with initial profile information of the patient obtained by at least one of (i) receiving the initial profile information through a web-based server website or (ii) by executing calls for the initial profile information to an application programming interface (API). The processor may be further configured to provide a verification code to the patient to verify a piece of contact information of the initial profile information, receive a return verification code from the patient, and complete registering the patient for access if a match between the provided and received verification codes is detected upon comparison of the provided and received verification codes. If a match between the provided and received verification codes is not detected, the processor may be configured to end the method.

In some embodiments, login credentials include a validation code, a username, a password, and/or a two-factor authentication credential. Information pertaining to the patient may include identity documents or information therefrom, an insurance card or information therefrom, and a name of a medical provider. The medical provider may be a primary-care physician or a specialist. Validating the identity of the patient may further include accepting at least one of a government issued photographic identification, a photograph of the patient's face, and an insurance card. The processor may be further configured to capture an image of a patient's face with an imaging device, and to reconcile the captured image with a previously accepted photograph of the patient's face.

In some embodiments, obtaining the healthcare data includes accessing a record locator service (RLS) database; searching the RLS database, based on proximity to an address represented by address information pertaining to the patient, for providers associated with the patient; and identifying at least one provider, based on the searching, from which to obtain healthcare data pertaining to the patient.

In some embodiments, the healthcare data pertaining to the patient includes past insurance claim data or a name of a medical provider obtained therefrom, and past data from an EOB form or a name of a medical provider obtained therefrom. The obtained healthcare data, as insurance data, may be stored on at least one of the identity server, the user health database server, and combinations thereof. The querying entity may be a patient, a medical provider, or a medical researcher.

In some embodiments, the querying entity may be a patient, a medical provider, or a medical researcher. The processor may be further configured to establish a telemedicine session between the patient and the querying entity by confirming possession of the right of access form accepted by the patient and stored in a memory device of at least one of the identity server and the user health database server, and by obtaining informed consent from the patient prior to establishing the telemedicine session.

In some embodiments, the querying entity may be a medical researcher. The processor may be further configured to confirm possession of the right of access form accepted by the patient and stored in the memory device, and to obtain informed consent from the patient. The processor may be further configured to anonymize the obtained healthcare data of the patient and to include the anonymized healthcare data of the patient in a pool of healthcare data of multiple patients.

In some embodiments the processor may be configured to automatically cause the HIE to receive a query from itself for healthcare data pertaining to a patient, instead of receiving such a query from an external querying entity. The HIE may be thought of as the querying entity in these embodiments. The processor may be further configured to automatically relay the query for healthcare data from the HIE toward a possessing entity. The HIE may act as a querying entity, for example, during initial setup and onboarding of a patient profile, when it may be desirable to automatically acquire any available healthcare data for the patient from any connected possessing entity. The processor may be further configured to automatically update the PHR by periodically querying possessing entities via the HIE on behalf of the patient.

In an embodiment, the healthcare data pertaining to the patient may be obtained from a possessing entity via an alternative login process described as follows. In the alternative login process, the processor, having granted HIE system access to a patient, is configured to receive a selection, by the patient, of a possessing entity whose server the patient intends to access directly, for example, by logging in to a website of the possessing entity. The alternative login process continues with the processor being configured to redirect the patient to a login page of the possessing entity. Such redirection may employ authentication flows, such as, for example, OAuth or security assertion markup language (SAML). The processor may be further configured to enable the patient to log in to the server of the possessing entity, respond to a request from the possessing entity to consent to sharing of healthcare data, specify access levels and permissions to which the possessing entity is expected to adhere, and to enable the HIE system to receive the healthcare data from the possessing entity and to store the received healthcare data in a database associated with the HIE system. In some implementations of the alternative login process, the HIE system may store a key with which to secure future access to the server of the possessing entity such that the processor may automatically update the PHR by periodically querying possessing entities for new healthcare data via the HIE on behalf of the patient.

In some embodiments, the possessing entity may be an insurance provider or a medical provider. The possessing entity may be a server of the HIE. The server of the HIE may be the identity server. The server of the HIE may be the user health database server. The method may further comprise relaying the query from the querying entity to the possessing entity, or to at least one of the HIE, the API, and a gateway of the HIE. The query for healthcare data may be relayed from the querying entity toward the possessing entity via a gateway of the HIE, or via e-mail or fax.

In some embodiments, the obtained healthcare data may be received in a format such as, for example, PDF, HTML, JSON, FHIR, HL7, DICOM, JPEG, MP4, MOV, GIF, PNG, SNOMED CT, LOINC, RxNorm, XML, CDATA, TXT, TEXT, JPG, MP3, AVI, SVG, DOC, DOCX, XLS, XLSX, and raw binary. The healthcare data, or the indication of an error, may be obtained by providing the possessing entity with a secure hyperlink or uniform resource locator (URL) leading to a provider and payer portal of the HIE system.

In some embodiments, the processor may be configured to segment healthcare data into a categorized time series using machine learning. The healthcare data categories created by segmentation may include, for example, insurance claims, diagnosis, medication, procedure, provider, facility, laboratory values, allergies, immunizations, clinical images, and family history.

In some embodiments, a processor may be configured to classify the obtained healthcare data as structured data or unstructured data. The processor may be further configured to classify unstructured data as being of a known format or of an unknown format. The processor may be further configured to classify unstructured data of an unknown format as being of a document or study of a known type. The processor may be further configured to, prior to storing the obtained healthcare data, wrapping in a shell document the obtained healthcare data classified as unstructured data of unknown format of unknown document or study type. The shell document format may be, e.g., CDATA. The processor may be further configured to convert unstructured data of unknown format of known document or study type to structured data by performing optical character recognition (OCR) based on pattern recognition for known terms of study. The processor may be further configured to convert unstructured data of known format to structured data by performing OCR based on pattern recognition for terms presently stored on the user health database server. The processor may be further configured to, prior to storing the obtained healthcare data, converting, to a standard format, the obtained healthcare data classified as or converted to structured data. The standard format may be, e.g., FHIR.

In some embodiments, the processor may be configured to determine, based on at least one of the categorized time series created by segmentation, a health score for the patient. The processor may be further configured to classify a health condition of the patient based on the health score. The health condition may include an assessment of a likelihood of a patient to be dangerous or otherwise impactful to public health. The processor may be further configured to issue a certificate that attests to information of a patient including at least one of a PHR history, a health score, and a health condition. An aspect of a PHR history to which an issued certificate may attest may include, for example, an indication of test results and/or of a vaccination status, such as for SARS-CoV2 (COVID-19). The certificate may be a printable document. The certificate may include a URL, or a representation thereof, leading to a webpage enabling retrieval from the HIE of a copy of the certificate, or a representation thereof, for example, to demonstrate authenticity of the certificate.

In some embodiments, the processor may be configured to determine that a specified period of time has passed prior to obtaining the healthcare data from the possessing entity, and to prompt an administrator or a legal entity to send a legal demand letter regarding the healthcare data to the possessing entity on behalf of the patient.

In another embodiment, a system for managing access to patient healthcare data includes a processor and a memory device with instructions loaded thereon, the instructions, when loaded, configuring the processor to register a patient for access to an HIE system. The processor is further configured to provide login credentials to the patient. The processor is further configured to grant the patient access to the HIE system on an identity server by validating login credentials submitted by the patient against the provided login credentials. The processor is further configured to end the method or to allow the patient another login attempt if the login attempt fails. The processor is further configured to obtain, from the patient, information pertaining to the patient including an identity of the patient. The processor is further configured to validate the received identity of the patient. The processor is further configured to end the method if the patient identity is found to be invalid. The processor is further configured to generate a right of access form and provide the right of access form to the patient. The processor is further configured to obtain an accepted right of access form from the patient. The processor is further configured to store the right of access form in a memory device of at least one of the identity server and a user health database server. A right of access form may be understood to be a document allowing an individual, who is a subject of PHI, to invoke rights granted in 45 C.F.R. § 164.524, Access of individuals to protected health information. The right of access form may be a digital document. The processor is further configured to receive a query for healthcare data pertaining to the patient from a querying entity. The processor is further configured to relay the query for healthcare data toward a possessing entity. The processor is further configured to obtain the healthcare data, or an indication of an error, from the possessing entity through the HIE system. The processor is further configured to relay the obtained healthcare data, or the indication of an error, to the querying entity. The processor is further configured to create or augment a PHR by storing the obtained healthcare data in a memory device of at least one of the identity server and the user health database server. This embodiment may further optionally include any features described herein in connection with any of the other embodiments described herein.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A illustrates elements of an example system for managing access to patient healthcare data.

FIG. 1B illustrates elements of a solution website employed in an example system for managing access to patient healthcare data.

FIG. 1C illustrates elements of an example user health database module.

FIG. 1D illustrates elements of an example identity server module.

FIG. 2A is a flow diagram illustrating an example method of managing access to patient healthcare data.

FIG. 2B is a flow diagram illustrating a process of provider-initiated on-boarding of a patient into an example system for managing access to patient healthcare data.

FIG. 2C is a flow diagram illustrating a process of patient-initiated on-boarding of the patient into an example system for managing access to patient healthcare data.

FIG. 2D is a flow diagram illustrating a process of patient insurance data retrieval from an example patient healthcare database.

FIG. 2E is a flow diagram illustrating a process of patient medical data retrieval from an example patient healthcare database.

FIG. 3 is a flow diagram illustrating a process of populating an example patient healthcare database.

FIG. 4 is a flow diagram illustrating a process of preparing and adding patient healthcare data to an example database.

FIGS. 5A, 5B-13 are flow diagrams illustrating various embodiments of a method of managing access to patient healthcare data.

FIG. 14 illustrates an example computer network, over which, embodiments of the claimed systems and methods may operate.

FIG. 15 is a system block diagram illustrating an example computer network, over which, embodiments of the claimed systems and methods may operate.

### DETAILED DESCRIPTION

A description of example embodiments follows.

The claimed methods and systems allow individual patients to enforce their patient right of access under 45 CFR § 164.524, Access of individuals to protected health information. Under 45 CFR § 164.524, an individual has a right of access to inspect and obtain a copy of protected health information (PHI) about the individual in a designated record set, for as long as the PHI is maintained in the designated record set for providers or payers who have either administered treatment or been remitted payment for medical services rendered.

The claimed methods and systems enable a patient to use rights granted by 45 CFR § 164.524, to have their patient identity authenticated, to digitally sign a patient right of access form, and to automatically query systems of record for medical information using fax, email, or digital gateways. A right of access form may be understood to be a document allowing an individual, who is a subject of PHI, to invoke rights granted in 45 CFR § 164.524. The right of access form may be a digital document. The claimed methods and systems allow patients to extract multiple sets of provider-created information from various facilities, and to populate, both manually and automatically, a cloud-based secure database that transforms and loads patient health data into each individual patient's PHR. Data may be acquired in various formats, including, for example, PDF, HTML, JSON, FHIR, HL7, DICOM, JPEG, MP4, MOV, GIF, PNG, SNOMED CT, LOINC, RxNorm, XML, CDATA, TXT, TEXT, JPG, MP3, AVI, SVG, DOC, DOCX, XLS, XLSX, and raw binary. Patient health data may then be segmented, using machine learning, into discrete categories including diagnosis, procedure, medication, provider, facility, laboratory values, allergies, immunizations, clinical images, and family history.

Patients may be enabled to share records with providers, payers, researchers and family members at the patients' individual discretion. Data may be shared from within a PRM, which is a graphical user interface that has an anonymous mode and a consent authorized mode based on a user's credentials. The PRM may allow providers, payers, and researchers to administer their patient population pools. Authorized users, granted viewing access by the patient, may be enabled to view data stored in a patient's health record including patient identifying information (PII), while researchers may use the user interface to sort aggregated patient records that lack PII to create patient population pools based on variables of interest. The claimed methods and systems allow patient population pools to be aggregated for providers, payers and researchers. Since the law provides for patients to be made aware of specific reasons for requests to share health information, providers, payers, and researchers may create patient population pools of patients of interest for research, and may individually obtain consent of a patient for communications, without the patient's identity being revealed to the providers, payers, or researchers, until consent is obtained from the patient. Patients may decide to consent to revealing personally identifying information and to being contacted by the provider, payer, or researcher.

Currently systems of record and EMRs tend to be tied to a given data stack and data format, which prevents easy transfer of patient health information between different EMR database types. Competing EMRs use various different patient portals to allow access of a patient's health information electronically. As such, a patient who transfers institutions cannot easily transfer health information between provider systems due to issues of compatibility, as well as due to liability fears based on penalties associated with improper disclosure of personally identifying patient information, including the risk of a facility or a provider losing their license. While some application programing interfaces (APIs) exist, interoperability is limited to EMRs who have invested in designing these APIs to seamlessly transfer the patient health information between EMRs. The claimed method and system simplifies the health record transfer process by eliminating the need for manually emailing, mailing, or faxing third-party patient release forms that often are ignored at least for a significant period of time.

Medical records are often needed prior to a patient beginning a course of treatment and/or prior to reimbursement from insurance carriers or payers. By utilizing the patient right of access codified in 45 CFR § 164.524 and the associated patient right of access form, the claimed methods and systems allow the patient to digitally submit a demand to a possessing entity, if the possessing entity has not answered an outstanding query for healthcare data for a set period of time. A query for healthcare data may herein be referred to interchangeably as a request for healthcare data. The possessing entity may be a medical facility, a medical provider, or a payer for a designated record set (as defined in 45 CFR § 164.501). An insurance provider may herein be referred to interchangeably as a payer. The submitted demand may be in the form of a legal demand letter. A set period of time for response to this demand may be, e.g., 30 days.

PHRs, which may be presently aggregated and sorted, may be further processed by being anonymized, i.e., having PII thereof removed. Anonymized PHRs may then be presented to researchers. Researchers may apply filters using the system GUI to sort the PHRs so that researchers can curate and collate groups of individual PHRs that (i) match variables of interest and (ii) comply with HIPPA/HITECH laws and standards.

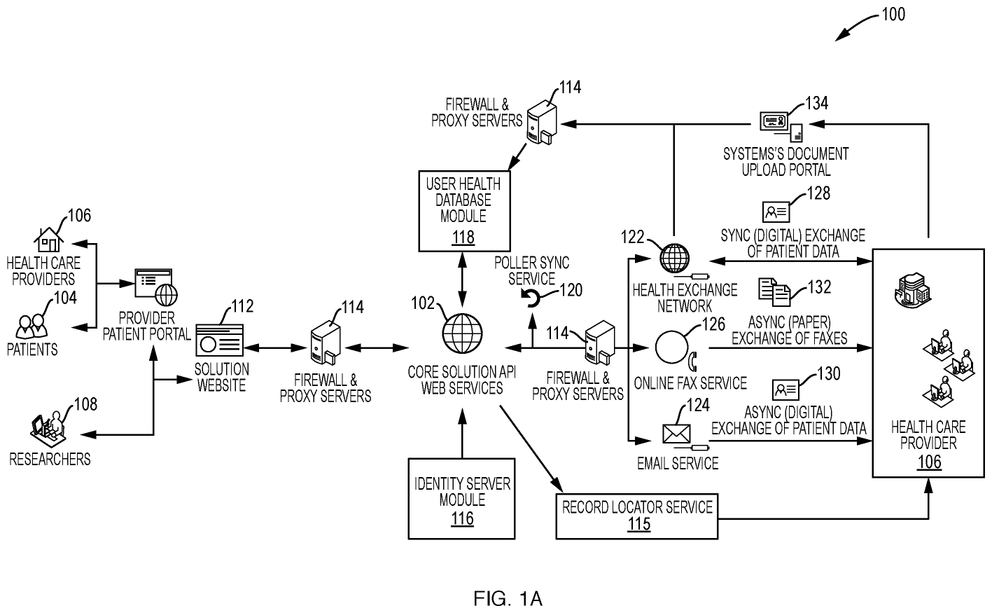

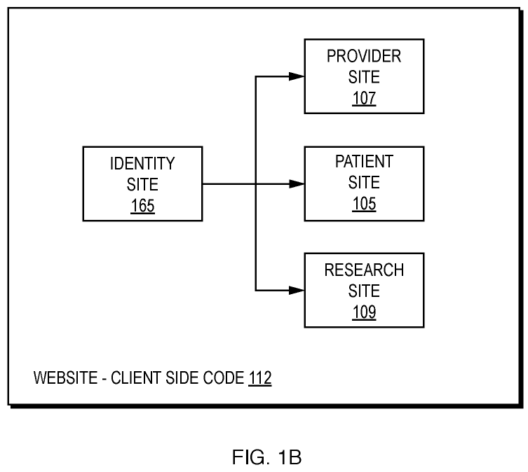

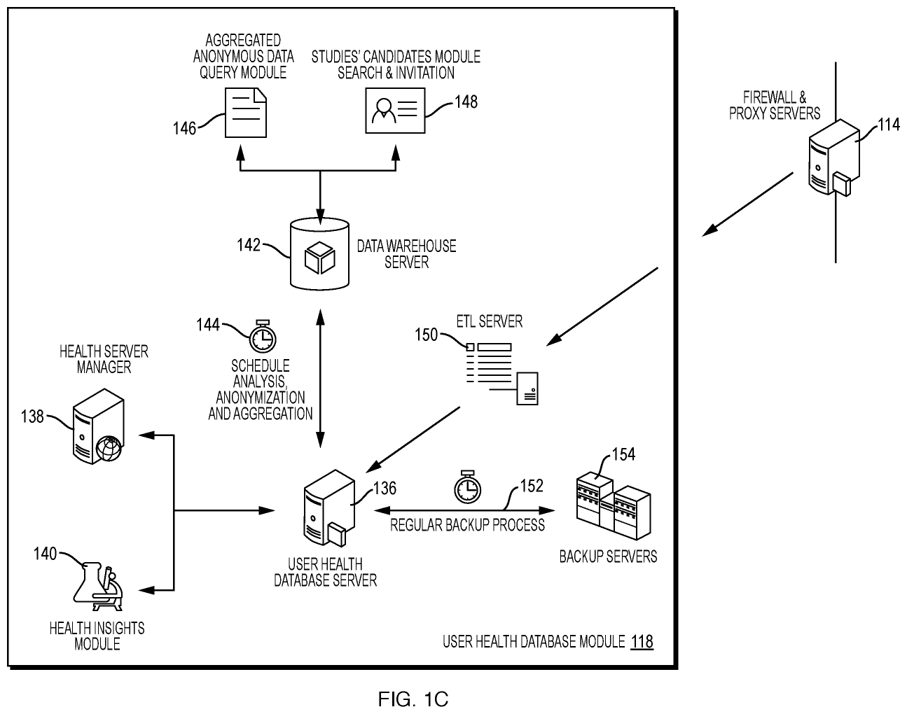

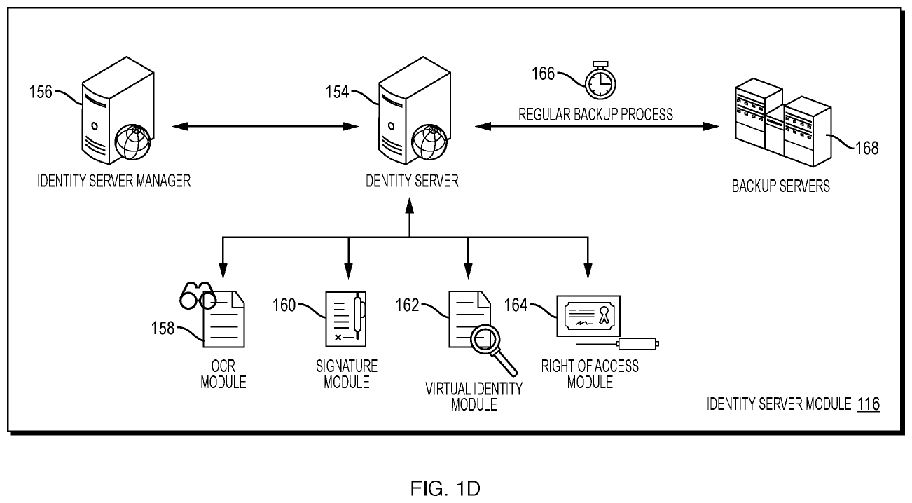

FIG. 1A depicts an example system 100 for managing access to patient healthcare data. An API enables control of various modules through a web service 102. Patients 104, health care providers 106, and medical researchers 108 may seek to access the system. Patients 104 may wish to take an active role in their personal healthcare, personally viewing and managing their healthcare data once the data has been gathered into a common repository by the system 100 and converted into a standard format compatible with the system 100. Healthcare providers 106 may include insurance providers, as well as providers of medical services. Healthcare data likewise may include insurance data, such as claims data or past explanations of benefits (EOBs), and healthcare data may also include records of medical care or medical information relating to the patient 104. Patients 104 and providers 106 may access the system 100 through a provider and patient portal 110, implemented on the solution website 112. Firewall and proxy servers 114 may be configured to regulate access to the API Web Services 102 by various modules of the system 100.

Continuing with respect to FIG. 1A, a record locator service (RLS) may be accessed via the API Web Services 102 to search an RLS database for information regarding a health care provider 106 of a patient 104. An RLS includes a local database 115 containing all zipcodes present in the country provided by official publicly accessible census and post office information, and corresponding latitude and longitude coordinates for each zip code. Such geo-referenced information provided by the RLS may be used to find health care providers 106 (e.g., medical providers, insurance companies, medical researchers, or any other entity that may hold health care records, insurance claims or documents of interest for a patient) associated with the patient 104. This is performed based at least on proximity, using, as inputs, addresses on file for the patient 104, as well as a configurable radius value in conjunction with the patient's 104 history.

To further describe FIG. 1A, an identity server module 116 may be configured to validate an identity of a patient 104, and may be further configured to manage the patient's 104 right of access. A user health database module 118 may be configured to manage procurement and storage of healthcare data pertaining to the patient 104. A poller sync service 120 may be configured to scan connected providers 106 for instances of new healthcare data pertaining to the patient 104, so the new data can be acquired and the PHR updated. An HIE 122 may be configured to securely transmit healthcare data and queries therefor between patients 104, providers 106, and other entities connected to the system 100, providing a synchronous digital exchange 128 of patient data. An HIE may herein be referred to interchangeably as a health exchange network or a healthcare network. Some providers may alternatively receive queries through an e-mail service 124 or a fax service 126, respectively utilizing an asynchronous digital exchange 130 and an asynchronous paper exchange 132 of patient data. A document upload portal 134 may be configured to securely transfer healthcare data pertaining to the patient 104 between providers 106 and the user health database module 118. The document upload portal may be referred to interchangeably herein as a provider and payer portal.

FIG. 1B depicts an example embodiment of a solution website 112. In an embodiment, patients 104, providers 106, and researchers 108 respectively interface with a patient site 105, a provider site 107, and a research site 109 in order to gain access to the system 100. An identity site 165 may be configured to verify an identity of a user such as a patient 104, provider 106, or researcher 108, and to grant access to the user.

FIG. 1C depicts an example embodiment of a user health database module 118. In an embodiment, a user health database server 136 may be configured to acquire and store healthcare data pertaining to the patient 104. A health server manager 138 may be a server internal to an API web service 102 with an internal website accessible by administrators of the API web service 102. The internal website may be accessible by employees of the API web service 102 by following security protocols. The internal website and health server manager 138 may be used to perform actions flagged as needing manual attention, including addressing healthcare records that are partially recognized by the system and require human approval, re-establishing connections to the HIE that time out or return an error, monitoring performance, and performing security validations.

A health insights module 140 may use machine learning and/or artificial intelligence (AI) based on case studies to assign a health score to the patient 104. The health score may be at least partially determined based on specific categories such as chronic disease. The health score may be at least partially determined as a generalized or unified value based on a calculator application or calculation method such as the atherosclerotic cardiovascular disease (ASCVD) risk calculator. The health insights module 140 may, based on the health score, be configured to classify a health condition of the patient 104. The health condition of the patient 104 may include an assessment of a likelihood of a patient 104 to be dangerous, or otherwise impactful, to public health. The health insights module may enable issuance of certificates that attest to information of the patient 104 including at least one of a PHR history, the health score, and the health condition. The certificates may be printable documents. The certificates may include a uniform resource locator (URL), or a representation thereof, leading to a webpage enabling retrieval from the HIE of copies of the certificates, or representations thereof, for example, to demonstrate authenticity of the certificates. A representation of a URL may include a QR code or a barcode. The health insights module 140 may forecast the patient's 104 health into the future and may suggest preventive actions to be taken in hopes of improving wellbeing and mitigating disease risks. The health insights module 140 may use machine learning techniques including logistic classifiers, XGBoost, and cost-sensitive models to assess predictors of risk that may impact health scores.

A data warehouse server 142 may be configured to obtain PHR data. The data warehouse server 142 may promote efficient storage, analysis, and use of the PHR by performing data cleansing, i.e., removing incomplete data and/or data that may not be useful. The data warehouse server 142 may further promote efficiency by standardizing data of disparate sources or formats to a common format. The data warehouse server 142 may remove PII values from database entries as needed, such as to aggregate data from multiple patients 104. The data warehouse server 142 may assign data values to various categories. Categories may include insurance claims, diagnosis, medication, procedure, provider, facility, laboratory values, allergies, immunizations, clinical images, and family history. Categories may include filters employable by a user performing a search, and measurements or dimensions to be applied in calculations of related parameters. The data warehouse server 142 may import PHR data to a pre-defined database model. The data warehouse server 142 may perform optimization processes on databases and may pre-calculate commonly desired statistics.

The user health database module 118 may be configured to perform scheduled analysis, anonymization, and aggregation 144 of data.

An aggregated anonymous data query module 146 may be a portal that enables providers 106 and researchers 108 to access the information from the data warehouse server 142. The aggregated anonymous data query module 146 may enable providers 106 and researchers 108 to perform searches and data analysis using the filters, measurements, and/or dimensions defined or calculated by the data warehouse server 142. The aggregated anonymous data query module 146 may enable providers 106 and researchers 108 to obtain data aggregated for multiple patients including statistical information and charts, with any PII having been removed by the data warehouse server 142.

A studies' candidates module 148 may be a portal module that enables researchers 108 to reach out to patients 104 to invite patients 104 to clinical trials or other relevant studies while PII of the patients 104 remains private. Patients 104 may receive a formal invitation with benefits and obligations of participation in a clinical trial explicitly stated on the invitation. The studies' candidates module 148 may provide patients 104 with a digital portal through which to affirm consent to the stated obligations and benefits. The patients 104 may also, though the digital portal, affirm consent to be contacted directly by a researcher 108. The API web service 102 may facilitate contact between patients 104 and researchers 108. Since the data warehouse server 142 may not store PII, the studies' candidates module 148 may use hash values as references between data stored on the data warehouse server 142 and data stored on the user health database server 136 or the identity server 154 depicted on FIG. 1D.

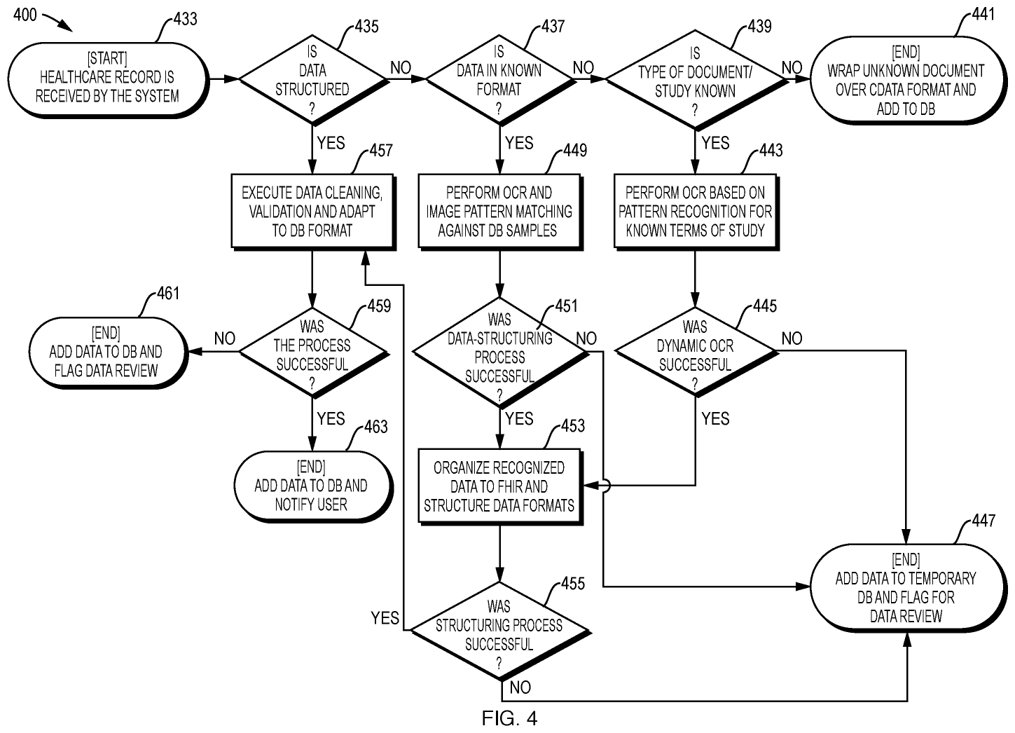

An ETL server 150 may be configured to perform actions directed to preparing and adding healthcare data to an example database as described below in reference to FIG. 4.

A regular backup process 152 may be configured to periodically back up data, stored on the user health database server 136, to at least one backup server 154. Backups of the data warehouse server 142 are not required, since information stored thereon can be re-generated from data stored on the user health database server 136.

FIG. 1D depicts an example embodiment of an identity server module 116. In an embodiment, an identity server 154 may be configured to verify an identity of an entity attempting to access the system 100.

An identity server manager 156 may be a server configured to provide a portal for entities such as system administrators, security personnel, and information technology personnel to connect to the identity server 154 to manage administrative, security, and information technology tasks. The identity server manager may enable creation and/or use of user management profiles, API security tokens, and general configuration tasks.

An OCR module 158 may detect text in non-structured healthcare data files. Text may be presented within an image, and the OCR module 158 may be configured to return a character representation of the text present in that image. Text produced by the OCR module 158 may be accurate to a given degree of certainty, which may be presented as a percentage value. The OCR module 158 may take inputs including a document type, document structure, and images of similar document types to increase the recognition accuracy. The OCR module may be called by the ETL server 150 to perform health care record recognition and classification. The OCR module 158 may be called by the identity server 154 to facilitate onboarding of a patient 104, for example, to obtain documents including the patient's 104 driver's license and insurance card. The OCR module 158 may recognize text, bar codes, and/or quick response (QR) codes.

In some embodiments, an identity of a patient 104 is obtained via one or more identity documents, such as a driver's license as mentioned hereinabove. The processor may be configured to validate the obtained identity documents by interfacing with an external API. Such an external API may be hosted in an official capacity by an office with a recognized authority to maintain records identifying the patient, such as by a government office. The processor may be configured to interface with the API via a secure connection to affirm information presented within the obtained identity documents. Such affirming may include determining if the information is true and up to date. Such interfacing may include transferring of information, wherein the information is encoded for increased security, in order to protect the privacy of the patient 104. For example, the OCR module 158 may recognize a bar code on a driver's license belonging to a patient 104, call a government API by decoding the bar code, and verify that the patient 104 information displayed on the driver's license is correct and current.

A signature module 160 may be configured to manage execution, or another form of acceptance, of a patient right of access form and possibly other pertinent documents. Such acceptance may be represented by a signed or otherwise executed instance of the right of access form, or may be represented via an electronic signature or other electronic indication of agreement.

A virtual identity module 162 may obtain text data recognized by the OCR module 158 and may cross-reference the text data with data stored in memory of a system server, such as the identity server 154 or the user health database server 136. For driver's license documents, the virtual identity module 162 may verify accuracy of data obtained from text and bar codes via the OCR module 158 against data stored on the identity server 154. The virtual identity module 162 may apply face pattern recognition techniques and machine learning to compare a photograph taken of a patient 104 in real time with the picture on the patient's 104 driver's license.

A right of access module 164 may be configured to manage the patient right of access form and execution thereof, and to maintain awareness as to the status thereof among various other modules of the system 100. A regular backup process 166 may be configured to periodically back up healthcare data on at least one backup server 168.

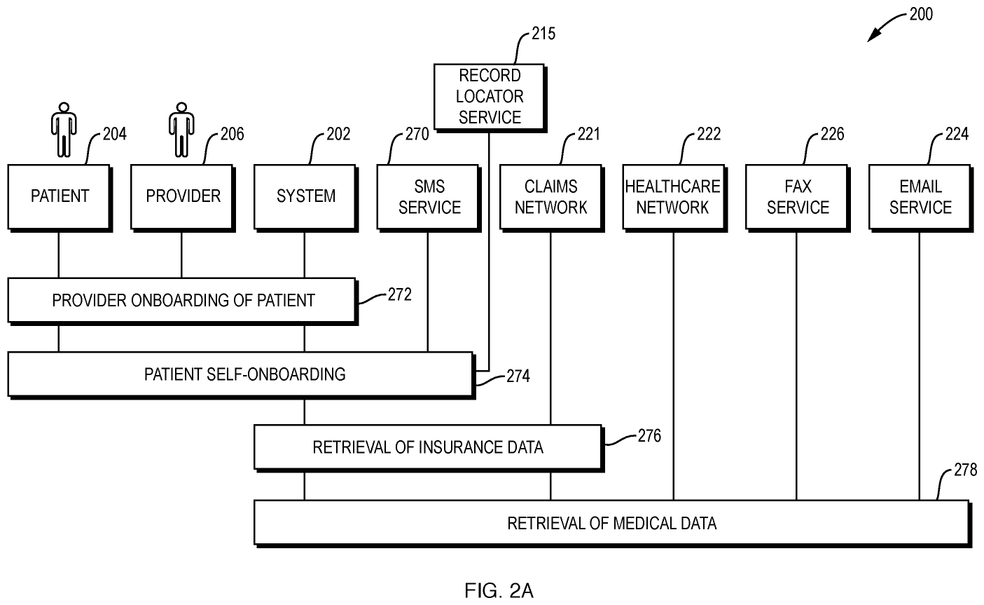

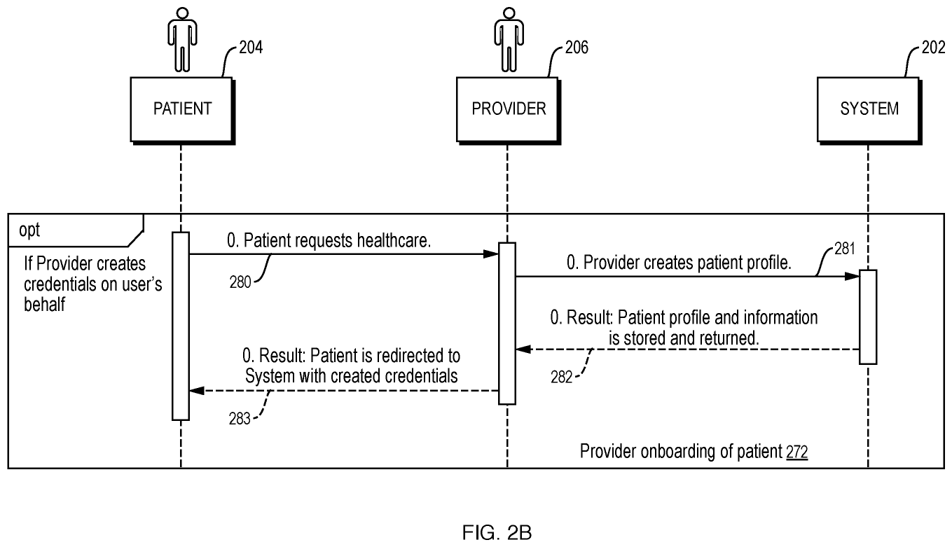

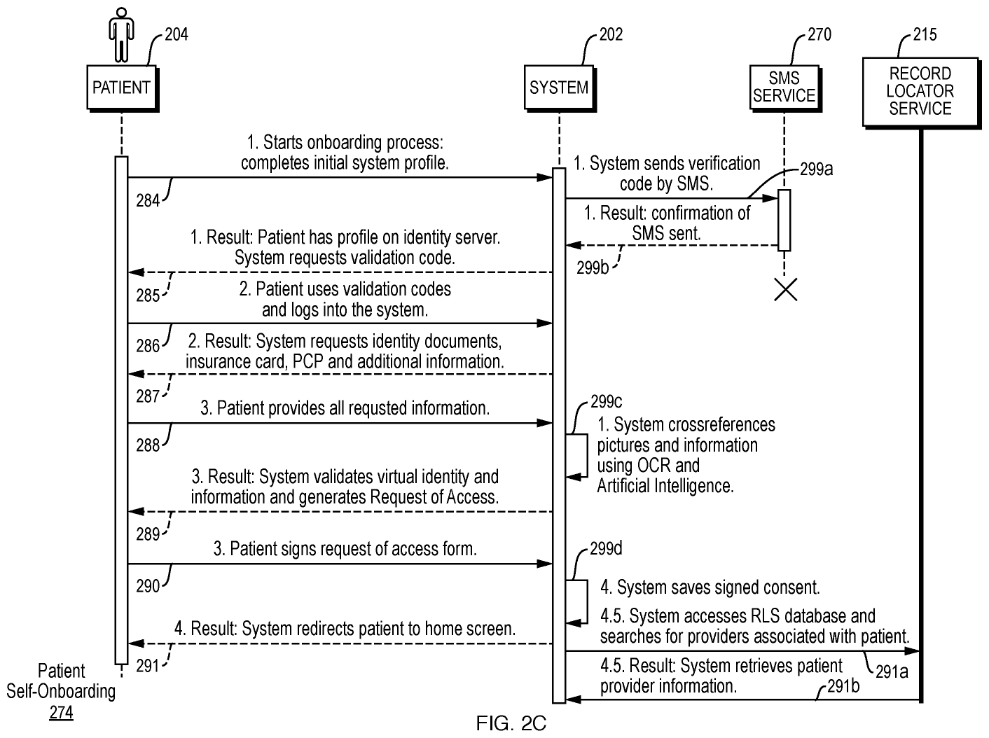

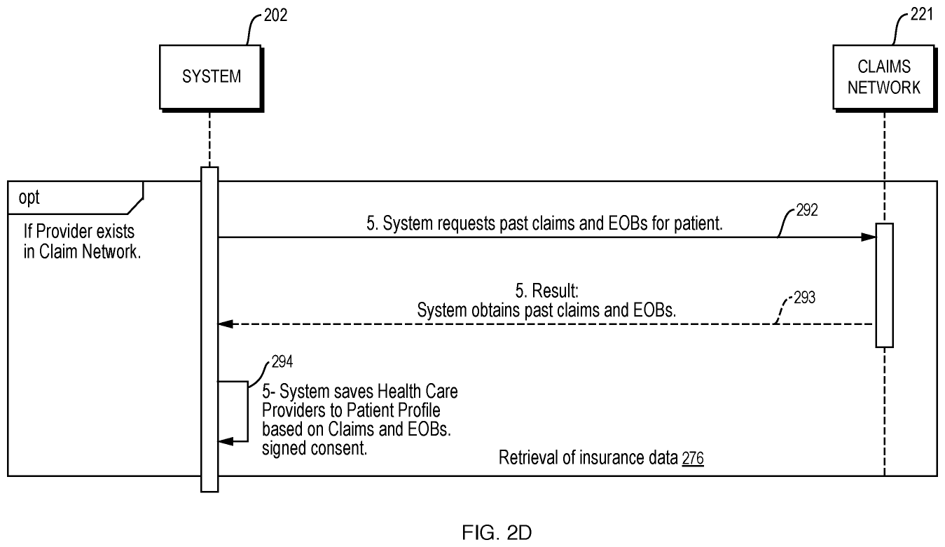

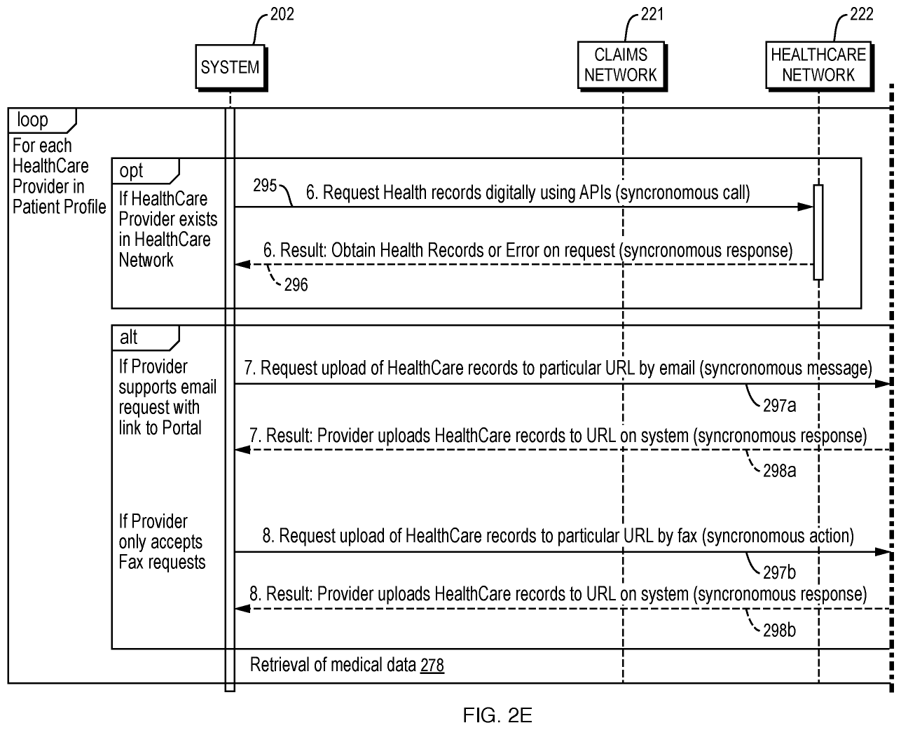

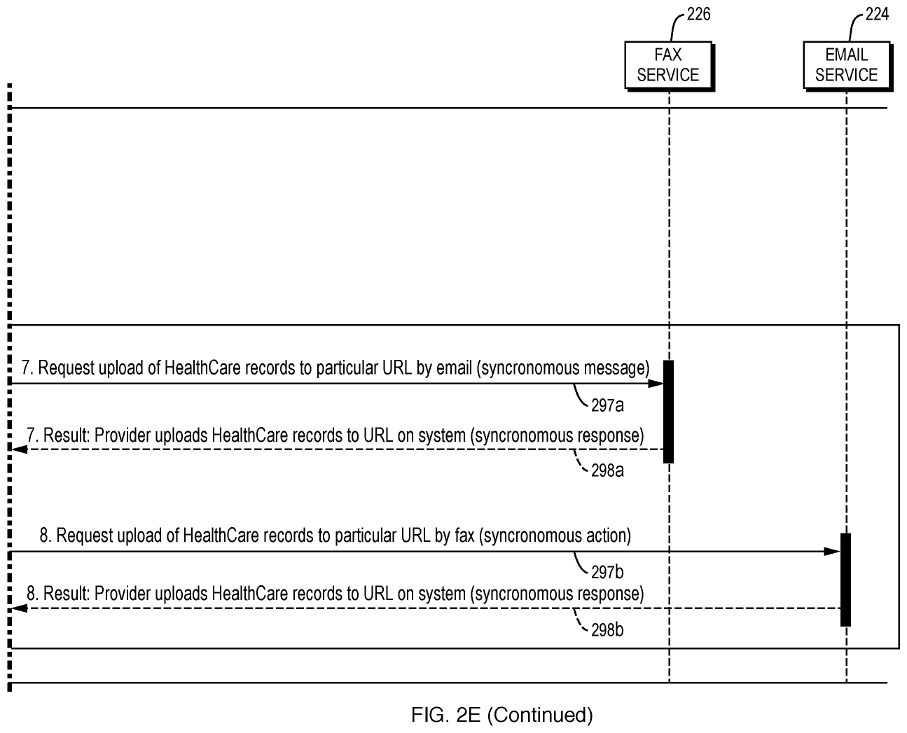

FIG. 2A illustrates an example method 200 for managing access to patient healthcare data. In some embodiments, an account may be established on the system 202 for a patient 204 through an onboarding process managed by the system 202. Optionally, the system 202 may be configured to interface with a patient 204 and a provider 206 and perform a provider onboarding 272 process. The system 202 may interface with a patient 204 and perform a patient self-onboarding 274 process, which may involve a text messaging or SMS service 270 and/or a record locator service 215. The system 202 may perform retrieval of insurance data 276 from a claims network 221. The insurance data may include a name of a medical provider from whom to procure medical data pertaining to the patient 204 in a bi-level querying process. Subsequent to or independent of a retrieval of insurance data 276, the system 202 may perform retrieval of medical data 278, interfacing with the claims network 221 and a healthcare network 222. The healthcare network may be an HIE 122 as described previously with reference to FIG. 1A. The system 202 may optionally connect with providers through an e-mail service 224 and/or a fax service 226 when issuing queries for data as part of the retrieval of medical data 278.

FIG. 2B illustrates an example embodiment of a provider onboarding process 272 for a patient 204. In an embodiment, a patient presents to a provider 206 requesting healthcare 280. The provider 206 may create a patient profile 281. The system 202 may store the patient profile 282, including information pertaining to the patient 204.

FIG. 2C illustrates an example embodiment of a patient self-onboarding process 274. In an embodiment, a patient 204 provides initial information 284 to establish a profile on the system 202. The system 202 may send a verification code 299b to a device controlled by the patient 204 via text or SMS service 270. The system 202 may receive a confirming response 299b from the patient 204 via SMS service 270. When the patient 204 wishes to proceed further, the system 202 may request a validation code 285 from the patient 204. The patient 204 may submit a validation code 286, and the system 202 may in turn permit the patient 204 to log in. The system 202 may request documents or related information 287 from the patient including identity documents, an insurance card, and/or a name of a provider. The patient 204 may provide information 288 to the system 202 to satisfy the aforementioned request 287. The system 202 may cross-reference 299c pictures and other information of the provided information 288 with information stored in memory of the system 202 using optical character recognition (OCR) and/or AI. The system may validate the patient's 204 identity and generate 289 a request of access form for the patient 204 to sign. The patient may sign said form 290, and the system may store 299d the signed form in memory. The system may then access an RLS database 291a and search the RLS database for providers 206 associated with the patient, based at least on proximity to address information of the patient 204. The system may then retrieve 291b information of at least one provider 206 associated with the patient 204. The system may then redirect the patient to a home screen 291.

FIG. 2D illustrates an example embodiment of an insurance data retrieval process 276. In an embodiment, the system 202 may request 292 insurance data including past claims and EOBs for the patient 204 from a claims network 221. The system may obtain 293 the past claims and EOBs. The system may verify the signature status of patient right of access and extract information regarding providers from the obtained 293 insurance data, and may save 294 information regarding the providers in memory associated with the patient profile.

FIG. 2E illustrates an example embodiment of a medical data retrieval process 278. In an embodiment, for each provider 206 in the patient profile, if the provider exists in the HIE 222 network, the system 202 requests health records digitally using APIs 295. The system 202 may obtain health records, or an error, in response 296 to the request 295. The system 202 may operate outside of the HIE network 222 by requesting 297a, via e-mail service 224, an upload of health care records to a location specified by a provided URL. The system 202 may receive 298a healthcare records uploaded to the URL location. Alternatively, the system 202 may request, 297b, via fax service, an upload of health care records to a provided URL. The system may receive 298b healthcare records uploaded to the URL location.

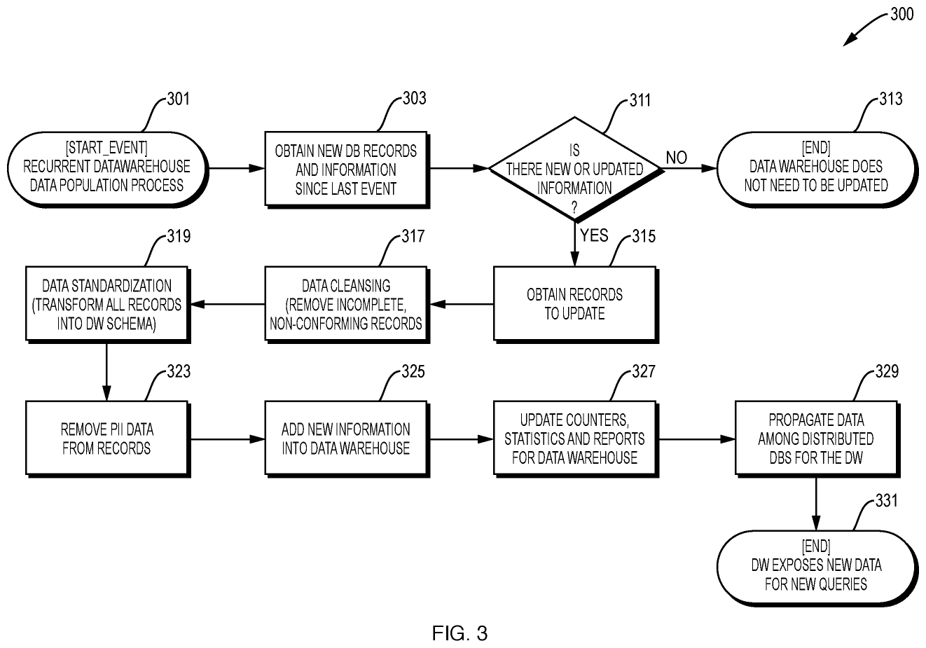

FIG. 3 illustrates an example embodiment of a process 300 for populating a data warehouse 142 for healthcare data. In an embodiment, the user health database module 118 is configured to issue a start event 301 for a data population process. The module 118 may obtain 303 new database records and information that has become available since a previous start event. The module 118 may check 311 for new or updated information. If no new or updated information exists, the process may end 313. If there is new or updated information, the module 118 may obtain 315 records to update. The module 118 may perform 317 data cleansing to remove incomplete and non-conforming records from the new database records. The module 118 may perform data standardization 319 to transform all new records into a format compatible with the data warehouse 142. The module 118 may add 325 the new records to the data warehouse 142. The module 118 may update counters, statistics, and reports 327 associated with the data warehouse 142. The module 118 may propagate 329 the new records among distributed data warehouses 142. The module 118 may end the process by configuring the data warehouses 142 to expose 331 the new data for new queries.

FIG. 4 illustrates an example embodiment of an ETL process, i.e., a process of preparing and adding patient healthcare data to an example database. In an embodiment, the user healthcare database module 118 receives 433 a new healthcare record. The module 118 may determine if the new record is made up of structured or unstructured data 435. If the new record is unstructured, the module 118 determines whether the new record is in a known format 437. If the new record is not in a known format, the module 118 determines if the new record is of a known document or study type 439. If the new record is not of a known document or study type, the module 118 may end the method 400 by wrapping 441 the new record in a shell document and storing the new record in memory. The format of the shell document may be, e.g., CDATA. If the new record is of a known document or study type, the module 118 may perform OCR 443 based on pattern recognition for known terms of study. The module may determine 445 if performance of OCR was successful. If OCR was not successful, the module 118 may end 447 the method 400, add the new record to a temporary database, and flag the new record for review. If OCR was successful, the module 118 may convert 453 the new record to a structured FHIR format.

In an embodiment, if the new record is of a known format, the module 118 may perform 449 OCR and image pattern matching against samples of data from memory to structure the data. The module 118 may determine 451 if structuring the data was successful. If structuring the data was not successful, the module 118 may end 447 the method 400, add the new record to a temporary database, and flag the new record for review. If structuring the data was successful, the module 118 may convert 453 the new record to a structured FHIR format.

In an embodiment, after structuring, the module may determine 455 if the structuring process was successful. If structuring the data was not successful, the module 118 may end 447 the method 400, add the new record to a temporary database, and flag the new record for review. If structuring the data was successful, or if the data was already structured as obtained, the module 118 may execute a process 457 of data cleaning, validation, and conversion to a format compatible with the database. If the process 457 was not successful, the module 118 may end 463 the method 400, store the new record in memory, and flag the new record for data review. If the process 457 was successful, the module 118 may end 461 the method 400, store the new record in memory, and notify a user.

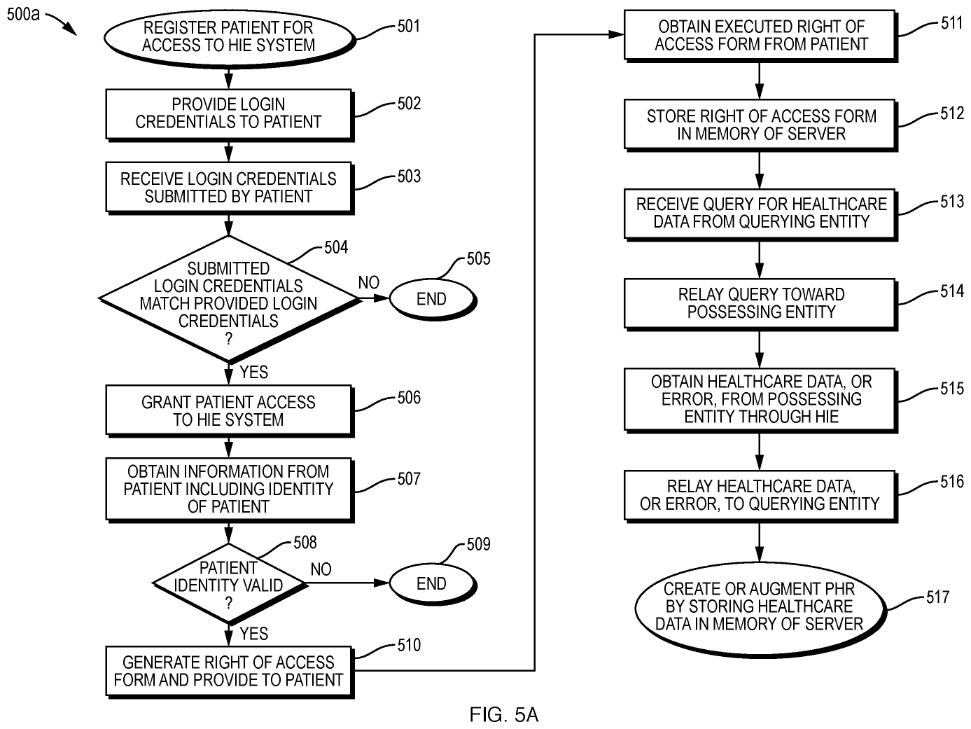

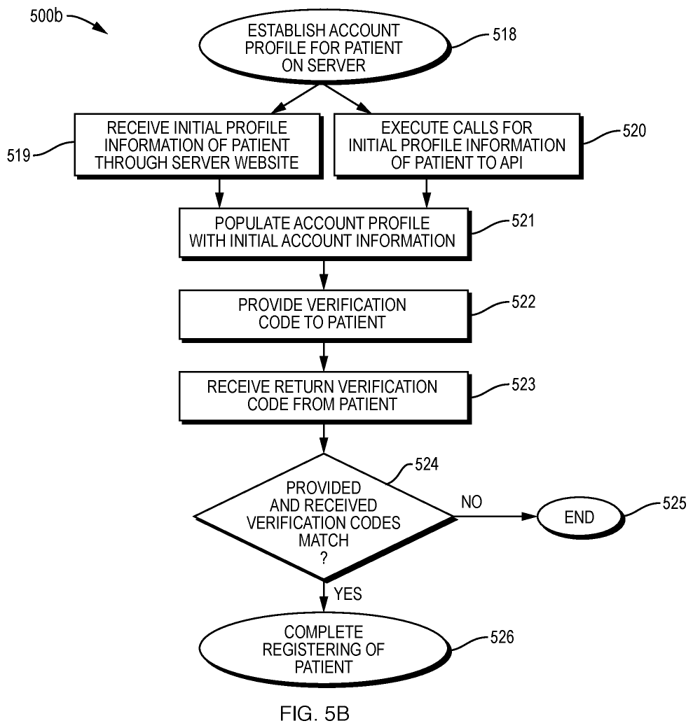

FIGS. 5A, 5B-13 show several example methods of managing access to patient healthcare data, selected to collectively show every aspect of the claimed methods and systems in an appropriate context. It should be noted that the examples presented herein are not limiting, and that other example methods of managing access to patient healthcare data may be realized through different combinations of the various elements shown in FIGS. 5A, 5B-13 and described herein.

FIG. 5A illustrates an embodiment of a method 500a of managing access to patient healthcare data. According to the embodiment, a processor may be configured to register 501 a patient for access to an HIE system. The processor may be further configured to provide login credentials 502 to the patient. The processor may be further configured to grant the patient access 506 to the HIE system on an identity server by validating login credentials 504 submitted 503 by the patient against the provided login credentials. The processor may be further configured to end 505 the method 500a or to allow (not pictured) the patient another login attempt if the login attempt fails. The processor may be further configured to obtain 507, from the patient, information pertaining to the patient including an identity of the patient. The processor may be further configured to validate 508 the received identity of the patient. The processor may be further configured to end 509 the method 500a if the patient identity is found to be invalid. The processor may be further configured to generate 510 a right of access form and provide the right of access form to the patient. The processor may be further configured to obtain 511 an executed right of access form from the patient. The processor may be further configured to store 512 the right of access form in a memory device of at least one of the identity server and a user health database server. The processor may be further configured to receive 513 a query for healthcare data pertaining to the patient from a querying entity. The processor may be further configured to relay 514 the query for healthcare data toward a possessing entity. The processor may be further configured to obtain 515 the healthcare data, or an indication of an error, from the possessing entity through the HIE system. The processor may be further configured to relay 516 the obtained healthcare data, or the indication of an error, to the querying entity. The processor may be further configured to create or augment 517 a PHR by storing the obtained healthcare data in a memory device of at least one of the identity server and the user health database server.

FIG. 5B illustrates an embodiment of a method 500b of registering a patient for access to an HIE system. According to the embodiment, a processor may be configured to establish 518, on the identity server, an account profile for the patient. The processor may be further configured to populate 521 the account profile with initial profile information of the patient obtained by at least one of (i) receiving 519 the initial profile information through a web-based server website or (ii) by executing 520 calls for the initial profile information to an API. The processor may be further configured to provide 522 a verification code to the patient to verify 524 a piece of contact information of the initial profile information, receive 523 a return verification code from the patient, and complete 526 registering the patient for access if a match between the provided and received verification codes is detected upon comparison of the provided and received verification codes. If a match between the provided and received verification codes is not detected, the processor may be configured to end 525 the method 500b.

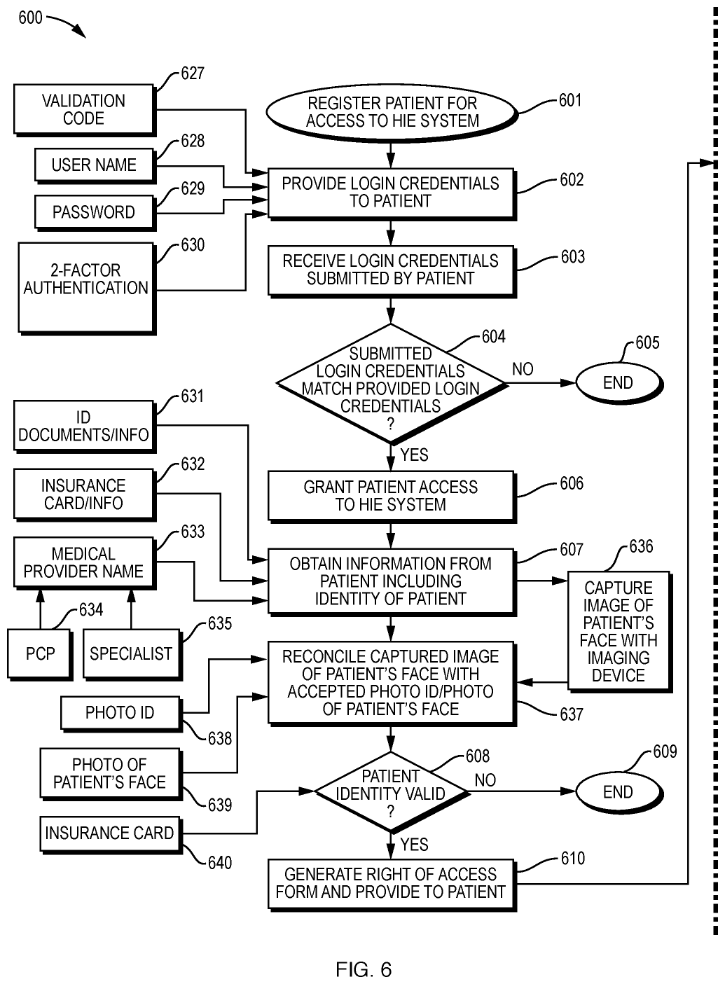

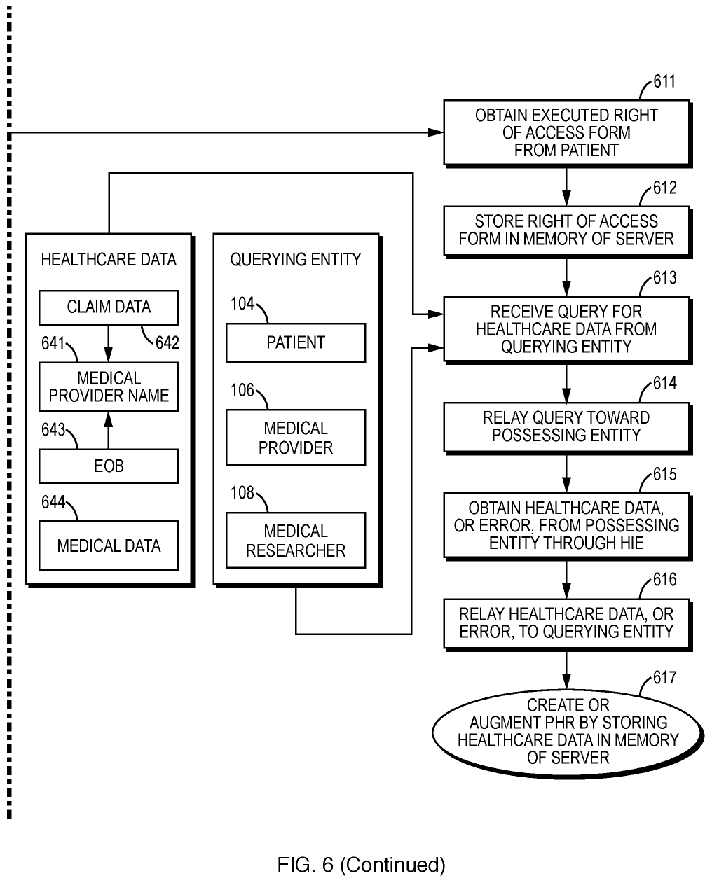

FIG. 6 illustrates an embodiment of a method 600 of managing access to patient healthcare data. According to the embodiment, a processor may be configured to register 601 a patient for access to an HIE system. The processor may be further configured to provide login credentials 602 to the patient. Login credentials may include a validation code 627, a username 628, a password 629, and/or a two-factor authentication credential 630. The processor may be further configured to grant the patient access 606 to the HIE system on an identity server by validating login credentials 604 submitted 603 by the patient against the provided login credentials. The processor may be further configured to end 605 the method 600 or to allow (not pictured) the patient another login attempt if the login attempt fails. The processor may be further configured to obtain 607, from the patient, information pertaining to the patient including an identity of the patient. Information pertaining to the patient may include identity documents or information therefrom 631, an insurance card or information therefrom 632, and a name of a medical provider 633. The medical provider may be a primary-care physician 634 or a specialist 635. The processor may be further configured to validate 608 the received identity of the patient. Validating 608 the identity of the patient may further include accepting at least one of a government issued photographic identification 638, a photograph of the patient's face 639, and an insurance card 640. The processor may be further configured to capture an image 636 of a patient's face with an imaging device, and to reconcile the captured image with a previously accepted photograph of the patient's face 637. The processor may be further configured to end 609 the method 600 if the patient identity is found to be invalid. The processor may be further configured to generate 610 a right of access form and provide the right of access form to the patient. The processor may be further configured to obtain 611 an executed right of access form from the patient. The processor may be further configured to store 612 the right of access form in a memory device of at least one of the identity server and a user health database server. The processor may be further configured to receive 613 a query for healthcare data pertaining to the patient from a querying entity. The healthcare data pertaining to the patient may include past insurance claim data 642 or a name of a medical provider 641 obtained therefrom, and past data from an EOB form 643 or a name of a medical provider 641 obtained therefrom. The obtained healthcare data, as insurance data, may be stored on at least one of the identity server, the user health database server, and combinations thereof. The querying entity may be a patient 104, a medical provider 106, or a medical researcher 108. The processor may be further configured to relay 614 the query for healthcare data toward a possessing entity. The processor may be further configured to obtain 615 the healthcare data, or an indication of an error, from the possessing entity through the HIE system. The processor may be further configured to relay 616 the obtained healthcare data, or the indication of an error, to the querying entity. The processor may be further configured to create or augment 617 PHR by storing the obtained healthcare data in a memory device of at least one of the identity server and the user health database server.

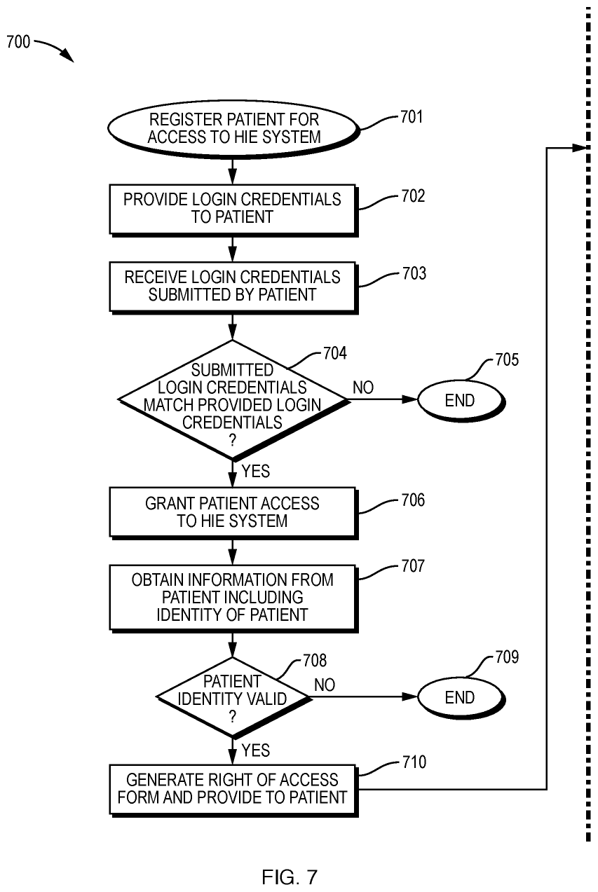

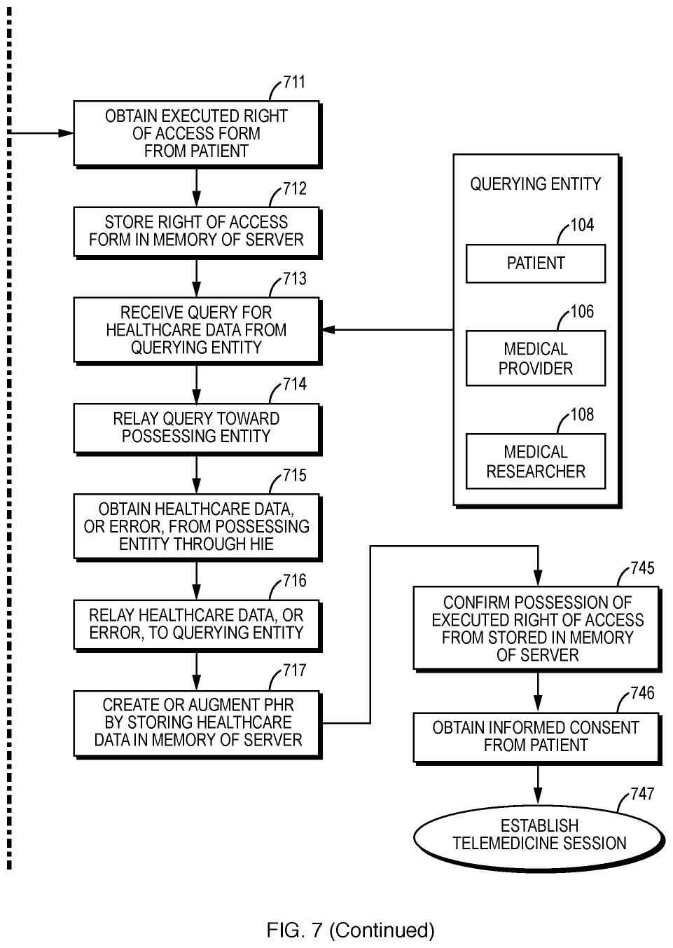

FIG. 7 illustrates an embodiment of a method 700 of managing access to patient healthcare data. According to the embodiment, a processor may be configured to register 701 a patient for access to an HIE system. The processor may be further configured to provide login credentials 702 to the patient. The processor may be further configured to grant the patient access 706 to the HIE system on an identity server by validating login credentials 704 submitted 703 by the patient against the provided login credentials. The processor may be further configured to end 705 the method 700 or to allow (not pictured) the patient another login attempt if the login attempt fails. The processor may be further configured to obtain 707, from the patient, information pertaining to the patient including an identity of the patient. The processor may be further configured to validate 708 the received identity of the patient. The processor may be further configured to end 709 the method 700 if the patient identity is found to be invalid. The processor may be further configured to generate 710 a right of access form and provide the right of access form to the patient. The processor may be further configured to obtain 711 an executed right of access form from the patient. The processor may be further configured to store 712 the right of access form in a memory device of at least one of the identity server and a user health database server. The processor may be further configured to receive 713 a query for healthcare data pertaining to the patient from a querying entity. The querying entity may be a patient 104, a medical provider 106, or a medical researcher 108. The processor may be further configured to relay 714 the query for healthcare data toward a possessing entity. The processor may be further configured to obtain 715 the healthcare data, or an indication of an error, from the possessing entity through the HIE system. The processor may be further configured to relay 716 the obtained healthcare data, or the indication of an error, to the querying entity. The processor may be further configured to create or augment 717 a PHR by storing the obtained healthcare data in a memory device of at least one of the identity server and the user health database server. The processor may be further configured to establish 747 a telemedicine session between the patient and the querying entity by confirming possession 745 of the right of access form executed by the patient and stored in a memory device of at least one of the identity server and the user health database server, and by obtaining informed consent 746 from the patient prior to establishing the telemedicine session.

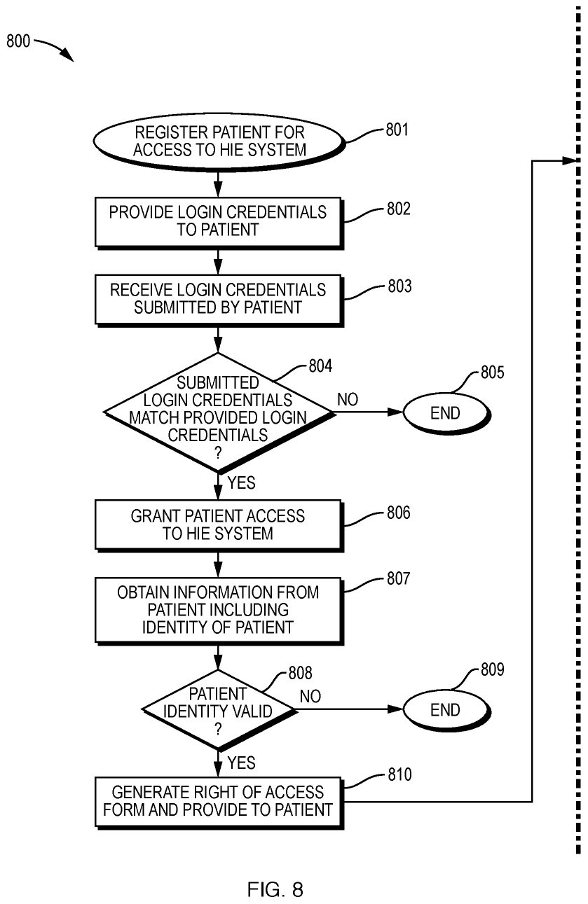

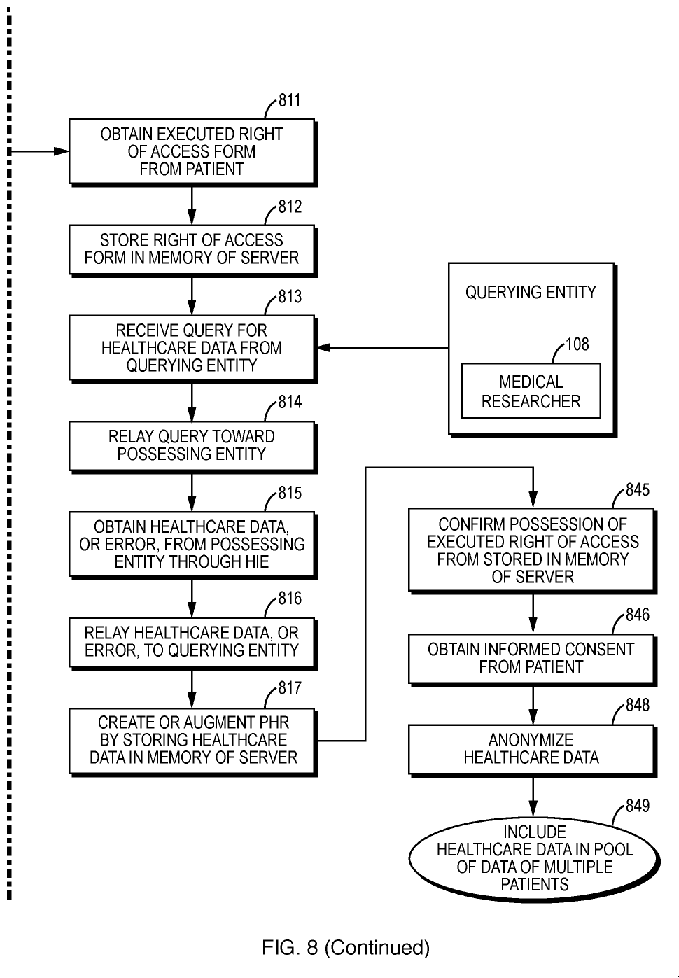

FIG. 8 illustrates an embodiment of a method 800 of managing access to patient healthcare data. According to the embodiment, a processor may be configured to register 801 a patient for access to an HIE system. The processor may be further configured to provide login credentials 802 to the patient. The processor may be further configured to grant the patient access 806 to the HIE system on an identity server by validating login credentials 804 submitted 803 by the patient against the provided login credentials. The processor may be further configured to end 805 the method 800 or to allow (not pictured) the patient another login attempt if the login attempt fails. The processor may be further configured to obtain 807, from the patient, information pertaining to the patient including an identity of the patient. The processor may be further configured to validate 808 the received identity of the patient. The processor may be further configured to end 809 the method 800 if the patient identity is found to be invalid. The processor may be further configured to generate 810 a right of access form and provide the right of access form to the patient. The processor may be further configured to obtain 811 an executed right of access form from the patient. The processor may be further configured to store 812 the right of access form in a memory device of at least one of the identity server and a user health database server. The processor may be further configured to receive 813 a query for healthcare data pertaining to the patient from a querying entity. The querying entity may be a medical researcher 108. The processor may be further configured to relay 814 the query for healthcare data toward a possessing entity. The processor may be further configured to obtain 815 the healthcare data, or an indication of an error, from the possessing entity through the HIE system. The processor may be further configured to relay 816 the obtained healthcare data, or the indication of an error, to the querying entity. The processor may be further configured to create or augment 817 a PHR by storing the obtained healthcare data in a memory device of at least one of the identity server and the user health database server. The processor may be further configured to confirm possession 845 of the right of access form executed by the patient and stored in the memory device, and to obtain informed consent 846 from the patient. The processor may be further configured to anonymize the obtained healthcare data of the patient 848 and to include the anonymized healthcare data of the patient in a pool of healthcare data of multiple patients 849.

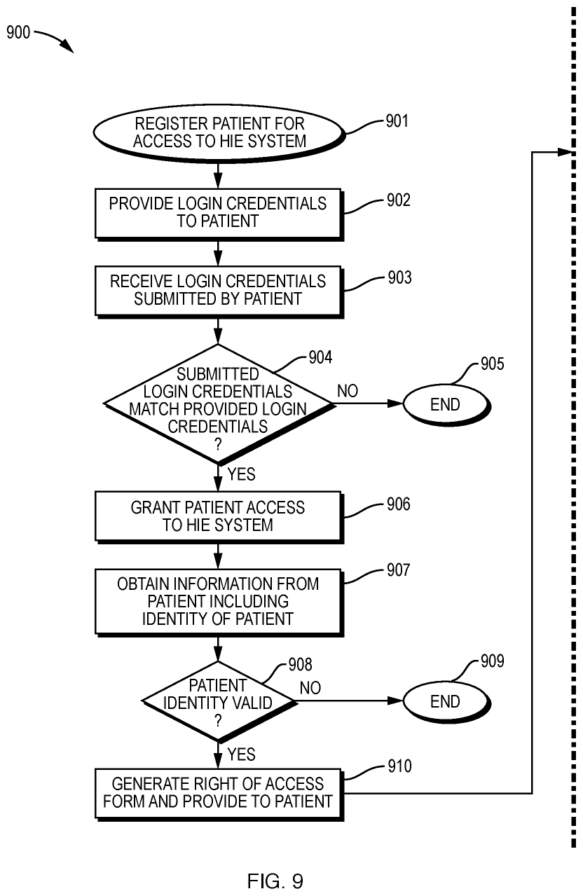

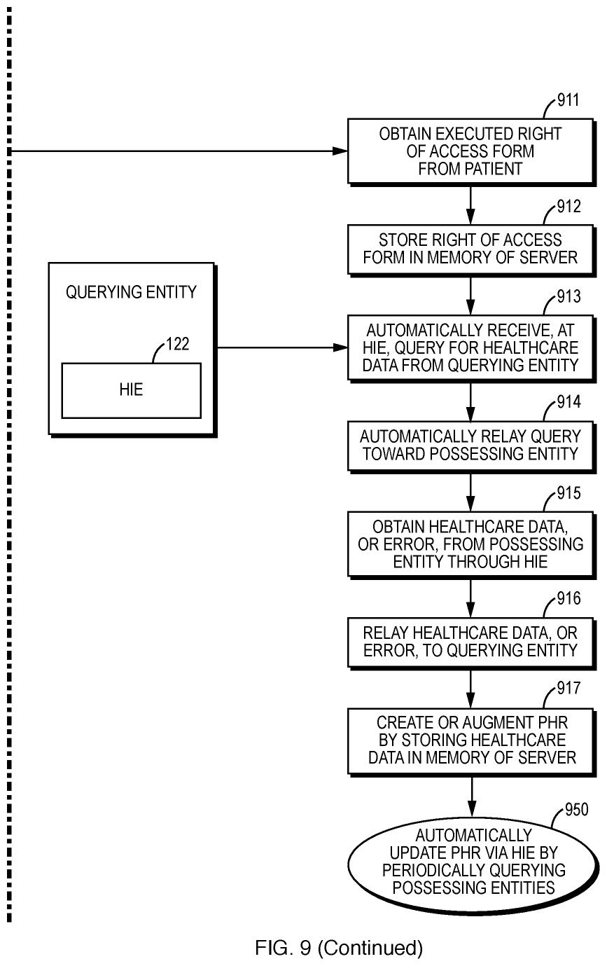

FIG. 9 illustrates an embodiment of a method 900 of managing access to patient healthcare data. According to the embodiment, a processor may be configured to register 901 a patient for access to an HIE system. The processor may be further configured to provide login credentials 902 to the patient. The processor may be further configured to grant the patient access 906 to the HIE system on an identity server by validating login credentials 904 submitted 903 by the patient against the provided login credentials. The processor may be further configured to end 905 the method 900 or to allow (not pictured) the patient another login attempt if the login attempt fails. The processor may be further configured to obtain 907, from the patient, information pertaining to the patient including an identity of the patient. The processor may be further configured to validate 908 the received identity of the patient. The processor may be further configured to end 909 the method 900 if the patient identity is found to be invalid. The processor may be further configured to generate 910 a right of access form and provide the right of access form to the patient. The processor may be further configured to obtain 911 an executed right of access form from the patient. The processor may be further configured to store 912 the right of access form in a memory device of at least one of the identity server and a user health database server. The processor may be further configured to automatically receive 913, at the HIE 122, a query for healthcare data pertaining to the patient from a querying entity. The querying entity may be the HIE 122. The processor may be further configured to automatically relay 914 the query for healthcare data toward a possessing entity. The processor may be further configured to obtain 915 the healthcare data, or an indication of an error, from the possessing entity through the HIE system. The processor may be further configured to relay 916 the obtained healthcare data, or the indication of an error, to the querying entity. The processor may be further configured to create or augment 917 a PHR by storing the obtained healthcare data in a memory device of at least one of the identity server and the user health database server. The processor may be further configured to automatically update 950 the PHR by periodically querying possessing entities on behalf of the patient.

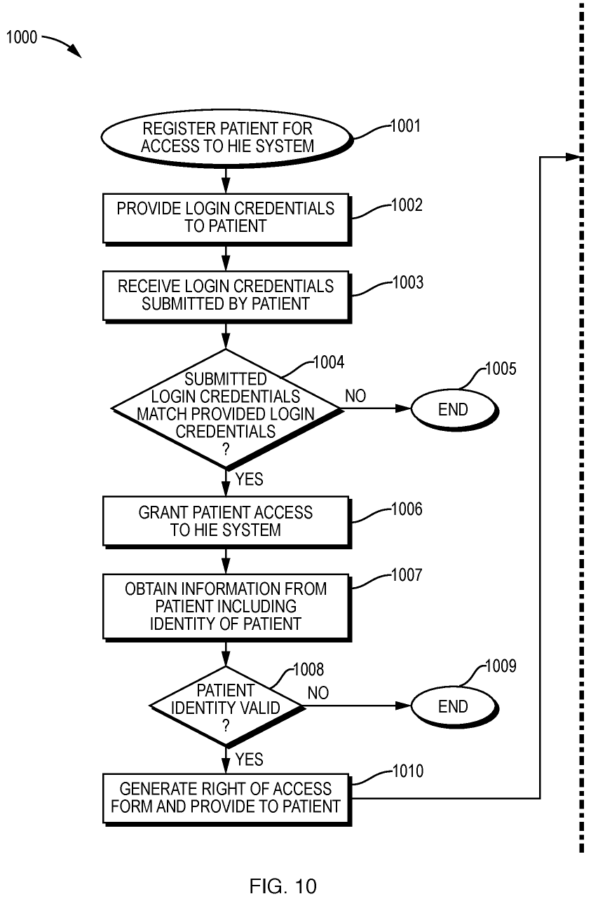

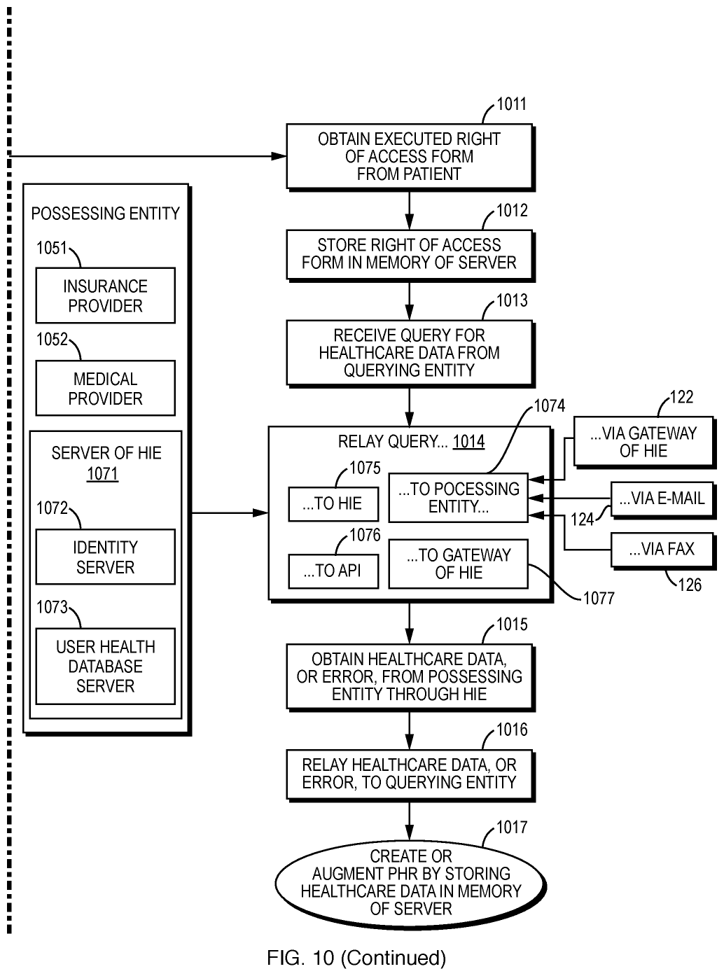

FIG. 10 illustrates an embodiment of a method 1000 of managing access to patient healthcare data. According to the embodiment, a processor may be configured to register 1001 a patient for access to an HIE system. The processor may be further configured to provide login credentials 1002 to the patient. The processor may be further configured to grant the patient access 1006 to the HIE system on an identity server by validating login credentials 1004 submitted 1003 by the patient against the provided login credentials. The processor may be further configured to end 1005 the method 1000 or to allow (not pictured) the patient another login attempt if the login attempt fails. The processor may be further configured to obtain 1007, from the patient, information pertaining to the patient including an identity of the patient. The processor may be further configured to validate 1008 the received identity of the patient. The processor may be further configured to end 1009 the method 1000 if the patient identity is found to be invalid. The processor may be further configured to generate 1010 a right of access form and provide the right of access form to the patient. The processor may be further configured to obtain 1011 an executed right of access form from the patient. The processor may be further configured to store 1012 the right of access form in a memory device of at least one of the identity server and a user health database server. The processor may be further configured to receive 1013 a query for healthcare data pertaining to the patient from a querying entity. The processor may be further configured to relay 1014 the query for healthcare data toward a possessing entity. The possessing entity may be an insurance provider 1051 or a medical provider 1052. The possessing entity may be a server 1071 of the HIE. The server 1071 may be the identity server 1072 or the user health database server 1073. The processor may be configured to relay the query from the querying entity to the possessing entity, or to at least one of the HIE, the API, and a gateway of the HIE. The query for healthcare data may be relayed from the querying entity toward the possessing entity via a gateway of the HIE 122, or via e-mail 124 or fax 126. The processor may be further configured to obtain 1015 the healthcare data, or an indication of an error, from the possessing entity through the HIE system. The processor may be further configured to relay 1016 the obtained healthcare data, or the indication of an error, to the querying entity. The processor may be further configured to create or augment 1017 a MR by storing the obtained healthcare data in a memory device of at least one of the identity server and the user health database server.

In some embodiments, the healthcare data pertaining to the patient may be obtained from a possessing entity via an alternative login process. In the alternative login process, the processor, having granted HIE system access to a patient, is configured to receive a selection, by the patient, of a possessing entity whose server the patient intends to access directly, for example, by logging in to a website of the possessing entity. The alternative login process continues with the processor being configured to redirect the patient to a login page of the possessing entity. Such redirection may employ authentication flows, such as, for example, OAuth or security assertion markup language (SAML). The processor may be further configured to enable the patient to log in to the server of the possessing entity, respond to a request from the possessing entity to consent to sharing of healthcare data, specify access levels and permissions to which the possessing entity is expected to adhere, and to enable the HIE system to receive the healthcare data from the possessing entity and to store the received healthcare data in a database associated with the HIE system. In some implementations of the alternative login process, the HIE system may store a key with which to secure future access to the server of the possessing entity such that the processor may automatically update the PHR by periodically querying possessing entities for new healthcare data via the HIE on behalf of the patient.

An example implementation of the alternative login process involves retrieval of healthcare data from an insurance provider. In the example implementation, the patient logs in to the HIE system using credentials that are validated via the identity server, as described hereinabove. Next, the system presents a list of insurance providers, from which the patient may select a provider with which the patient has established an account. The system may then redirect the patient to a login page of the selected insurance provider. Once the patient logs in to the selected insurance provider, the selected insurance provider asks the patient for consent to share her/his information with the HIE system. Once the patient agrees, and subsequently makes various selections pertaining to access levels, the insurance provider shares the appropriate healthcare data (e.g., EOBs and claims) with the HIE system.

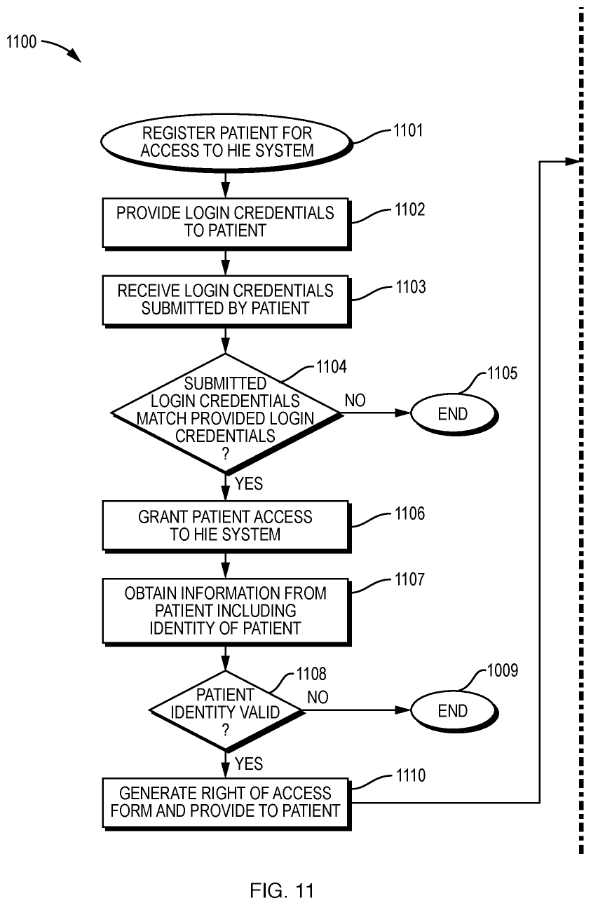

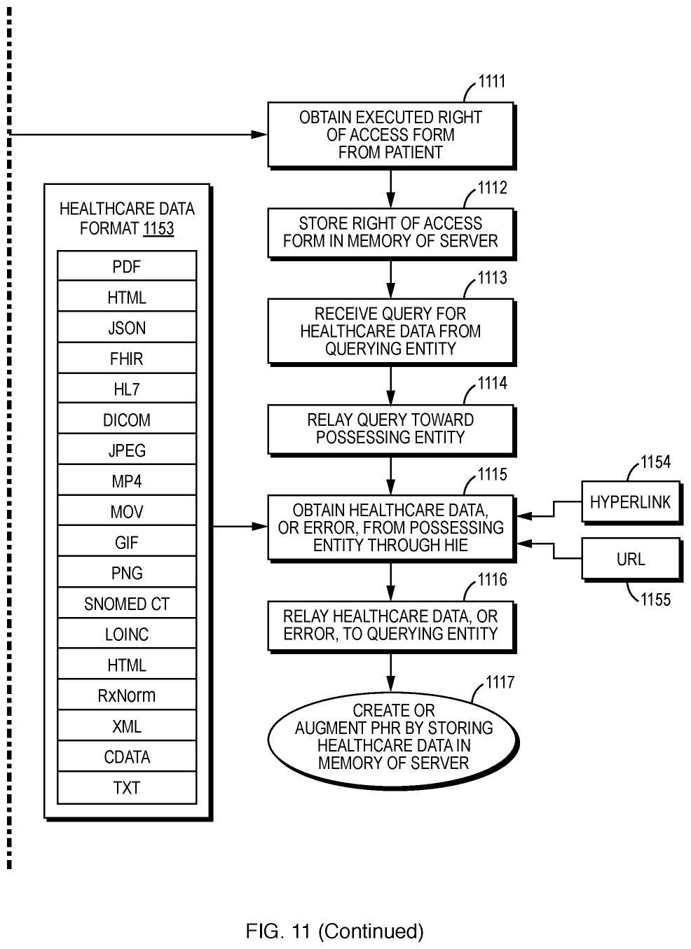

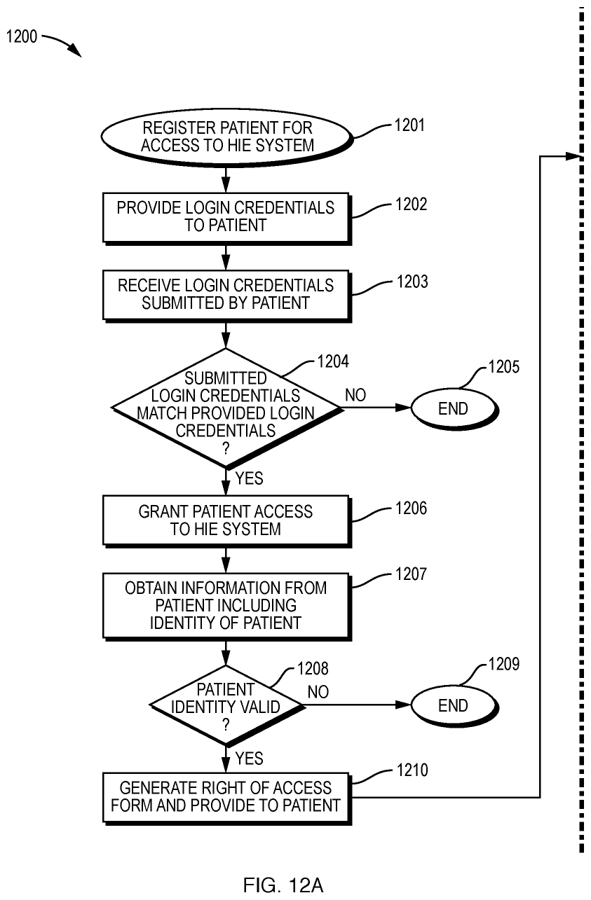

FIG. 11 illustrates an embodiment of a method 1100 of managing access to patient healthcare data. According to the embodiment, a processor may be configured to register 1101 a patient for access to an HIE system. The processor may be further configured to provide login credentials 1102 to the patient. The processor may be further configured to grant the patient access 1106 to the HIE system on an identity server by validating login credentials 1104 submitted 1103 by the patient against the provided login credentials. The processor may be further configured to end 1105 the method 1100 or to allow (not pictured) the patient another login attempt if the login attempt fails. The processor may be further configured to obtain 1107, from the patient, information pertaining to the patient including an identity of the patient. The processor may be further configured to validate 1108 the received identity of the patient. The processor may be further configured to end 1109 the method 1100 if the patient identity is found to be invalid. The processor may be further configured to generate 1110 a right of access form and provide the right of access form to the patient. The processor may be further configured to obtain 1111 an executed right of access form from the patient. The processor may be further configured to store 1112 the right of access form in a memory device of at least one of the identity server and a user health database server. The processor may be further configured to receive 1113 a query for healthcare data pertaining to the patient from a querying entity. The processor may be further configured to relay 1114 the query for healthcare data toward a possessing entity. The processor may be further configured to obtain 1115 the healthcare data, or an indication of an error, from the possessing entity through the HIE system. The obtained healthcare data may be received in a format 1153 such as, for example, PDF, HTML, JSON, FHIR, HL7, DICOM, JPEG, MP4, MOV, GIF, PNG, SNOMED CT, LOINC, RxNorm, XML, CDATA, TXT, TEXT, JPG, MP3, AVI, SVG, DOC, DOCX, XLS, XLSX, and raw binary. The healthcare data, or the indication of an error, may be obtained by providing the possessing entity with a secure hyperlink 1154 or URL 1155 leading to a provider and payer portal of the HIE system. Obtaining the healthcare data 1155 may include accessing a record locator service (RLS) database; searching the RLS database, based on proximity to an address represented by address information pertaining to the patient, for providers associated with the patient; and identifying at least one provider, based on the searching, from which to obtain healthcare data 1155 pertaining to the patient. The processor may be further configured to relay 1116 the obtained healthcare data, or the indication of an error, to the querying entity. The processor may be further configured to create or augment 1117 a PHR by storing the obtained healthcare data in a memory device of at least one of the identity server and the user health database server.

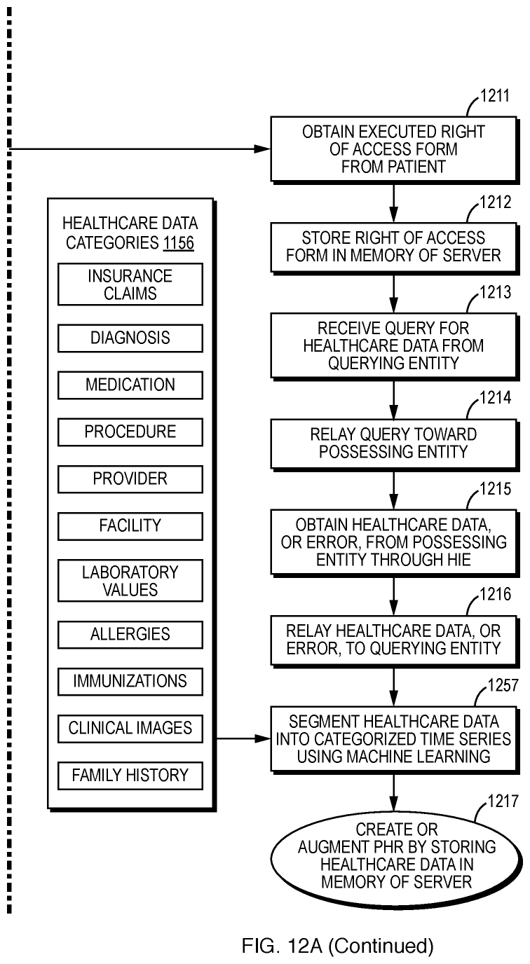

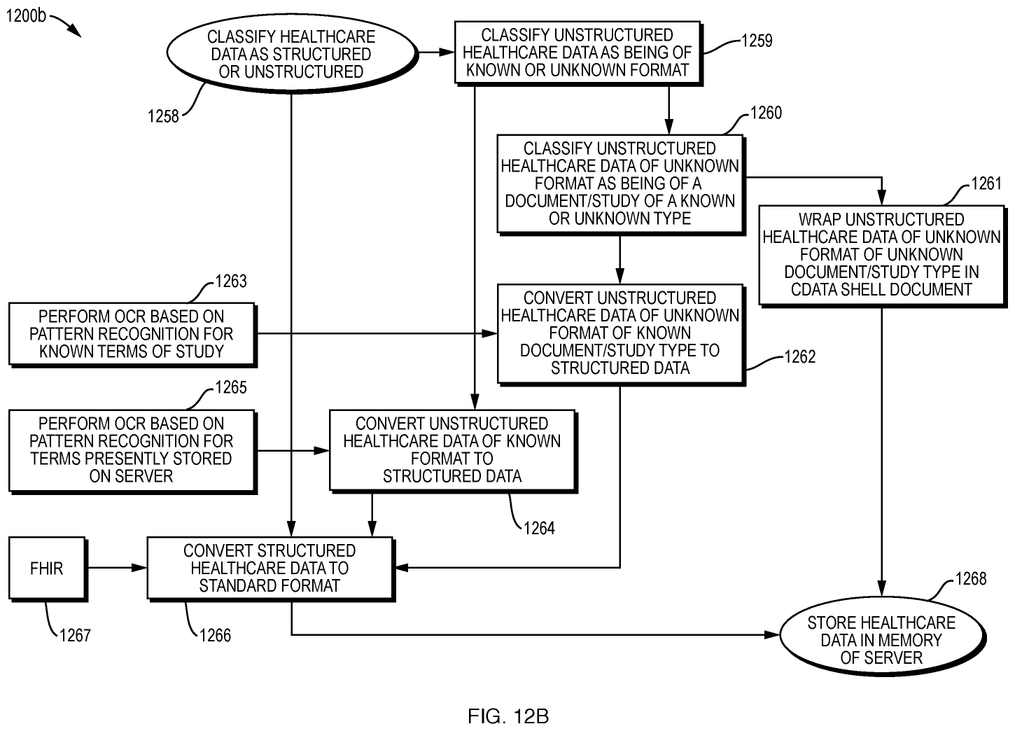

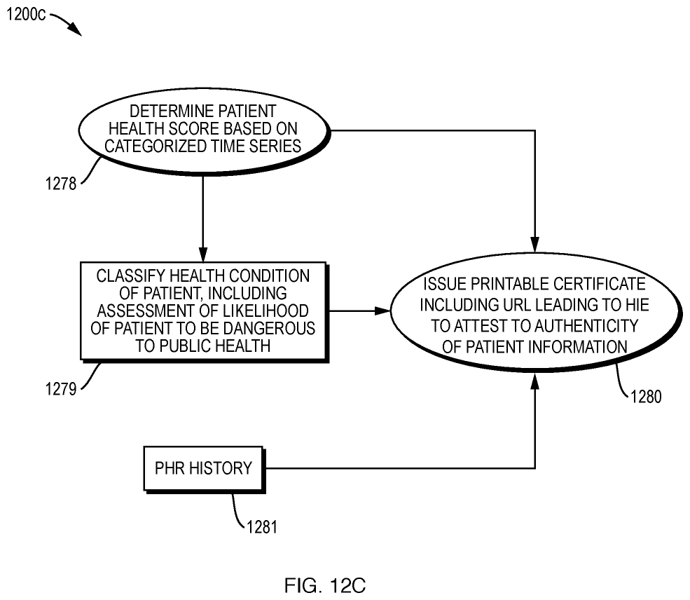

FIG. 12A illustrates an embodiment of a method 1200a of managing access to patient healthcare data. According to the embodiment, a processor may be configured to register 1201 a patient for access to an HIE system. The processor may be further configured to provide login credentials 1202 to the patient. The processor may be further configured to grant the patient access 1206 to the HIE system on an identity server by validating login credentials 1204 submitted 1203 by the patient against the provided login credentials. The processor may be further configured to end 1205 the method 1200a or to allow (not pictured) the patient another login attempt if the login attempt fails. The processor may be further configured to obtain 1207, from the patient, information pertaining to the patient including an identity of the patient. The processor may be further configured to validate 1208 the received identity of the patient. The processor may be further configured to end 1209 the method 1200a if the patient identity is found to be invalid. The processor may be further configured to generate 1210 a right of access form and provide the right of access form to the patient. The processor may be further configured to obtain 1211 an executed right of access form from the patient. The processor may be further configured to store 1212 the right of access form in a memory device of at least one of the identity server and a user health database server. The processor may be further configured to receive 1213 a query for healthcare data pertaining to the patient from a querying entity. The processor may be further configured to relay 1214 the query for healthcare data toward a possessing entity. The processor may be further configured to obtain 1215 the healthcare data, or an indication of an error, from the possessing entity through the HIE system. The processor may be further configured to relay 1216 the obtained healthcare data, or the indication of an error, to the querying entity. The processor may be further configured to segment 1257 healthcare data into categorized time series using machine learning. The healthcare data categories 1256 created by segmentation may include, for example, insurance claims, diagnosis, medication, procedure, provider, facility, laboratory values, allergies, immunizations, clinical images, and family history. The processor may be further configured to create or augment 1217 a PHR by storing the obtained healthcare data in a memory device of at least one of the identity server and the user health database server.

FIG. 12B illustrates an embodiment of a method 1200b for segmenting obtained healthcare data. According to the embodiment, the processor may be configured to classify 1258 the obtained healthcare data as structured data or unstructured data. The processor may be further configured to classify 1259 unstructured data as being of a known format or of an unknown format. The processor may be further configured to classify 1260 unstructured data of an unknown format as being of a document or study of a known type. The processor may be further configured to, prior to storing 1268 the obtained healthcare data, wrapping 1261 in a shell document the obtained healthcare data classified as unstructured data of unknown format of unknown document or study type. The format of the shell document may be, e.g., CDATA. The processor may be further configured to convert 1262 unstructured data of unknown format of known document or study type to structured data by performing optical character recognition (OCR) 1263 based on pattern recognition for known terms of study. The processor may be further configured to convert 1264 unstructured data of known format to structured data by performing OCR 1265 based on pattern recognition for terms presently stored on user health database server. The processor may be further configured to, prior to storing 1268 the obtained healthcare data, converting 1266, to a standard format, the obtained healthcare data classified as or converted to structured data. The standard format may be FHIR 1267.

FIG. 12C illustrates an embodiment of a method 1200c for classifying a condition of a patient based on segmented healthcare data pertaining to the patient. According to the embodiment, a processor may be configured to determine, based on at least one of the categorized time series created by segmentation 1257 as shown in FIG. 12A, a health score 1278 for the patient. The processor may be further configured to classify a health condition 1279 of the patient based on the health score. The health condition may include an assessment of a likelihood of a patient to be dangerous or otherwise impactful to public health. The processor may be further configured to issue a certificate 1280 that attests to information of a patient including at least one of a PHR history 1281, a health score 1278, and a health condition 1279. An aspect of a PHR history 1281 to which an issued certificate may attest may include, for example, an indication of test results and/or of a vaccination status, such as for SARS-CoV2 (COVID-19). The certificate 1280 may be a printable document. The certificate 1280 may include a URL, or a representation thereof, leading to a webpage enabling retrieval from the HIE of a copy of the certificate, or a representation thereof, for example, to demonstrate authenticity of the certificate. A representation of a URL may include a QR code or a barcode.

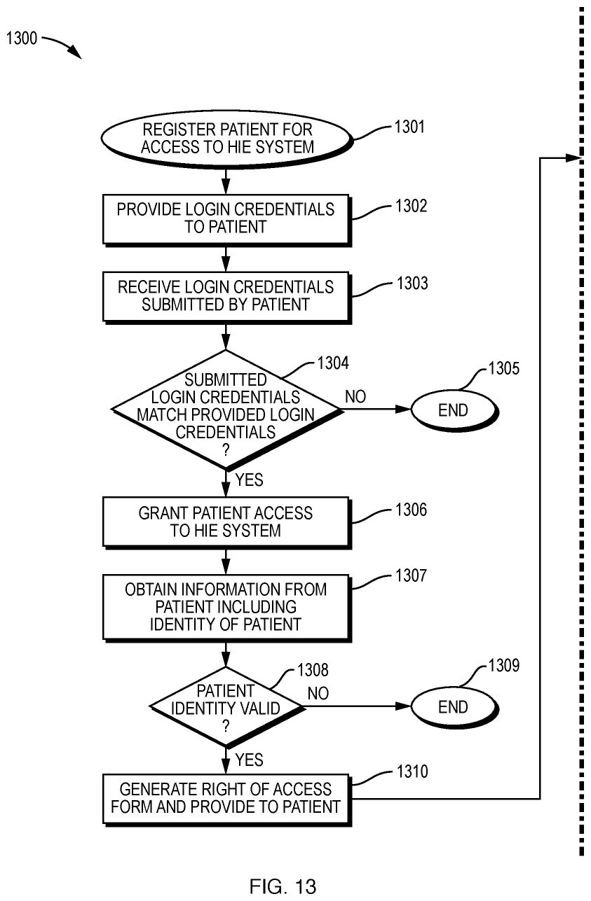

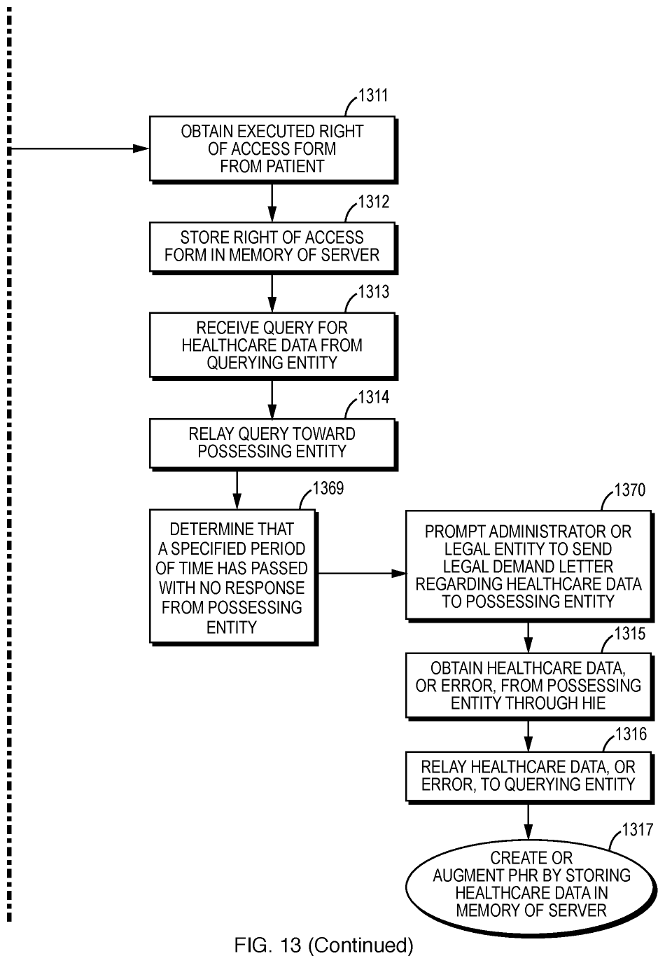

FIG. 13 illustrates an embodiment of a method 1300 of managing access to patient healthcare data. According to the embodiment, a processor may be configured to register 1301 a patient for access to a HIE system. The processor may be further configured to provide login credentials 1302 to the patient. The processor may be further configured to grant the patient access 1306 to the HIE system on an identity server by validating login credentials 1304 submitted 1303 by the patient against the provided login credentials. The processor may be further configured to end 1305 the method 1300 or to allow (not pictured) the patient another login attempt if the login attempt fails. The processor may be further configured to obtain 1307, from the patient, information pertaining to the patient including an identity of the patient. The processor may be further configured to validate 1308 the received identity of the patient. The processor may be further configured to end 1309 the method 1300 if the patient identity is found to be invalid. The processor may be further configured to generate 1310 a right of access form and provide the right of access form to the patient. The processor may be further configured to obtain 1311 an executed right of access form from the patient. The processor may be further configured to store 1312 the right of access form in a memory device of at least one of the identity server and a user health database server. The processor may be further configured to receive 1313 a query for healthcare data pertaining to the patient from a querying entity. The processor may be further configured to relay 1314 the query for healthcare data toward a possessing entity. The processor may be further configured to determine 1369 that a specified period of time has passed prior to obtaining the healthcare data from the possessing entity, and to prompt an administrator or a legal entity to send 1370 a legal demand letter regarding the healthcare data to the possessing entity on behalf of the patient. The processor may be further configured to obtain 1315 the healthcare data, or an indication of an error, from the possessing entity through the HIE system. The processor may be further configured to relay 1316 the obtained healthcare data, or the indication of an error, to the querying entity. The processor may be further configured to create or augment 1317 a PHR by storing the obtained healthcare data in a memory device of at least one of the identity server and the user health database server.

The processor may be configured to authorize a querying entity to submit queries for healthcare data to the system. The processor may be configured to curate a list of registered querying entities on the identity server or on the user health database server.

The following is an example embodiment of a method for managing access to patient healthcare data. A system operator/clinic/payer/provider may optionally initiate the patient registration process, and send an email to the patient to register and complete a system account. FIGS. 2A-B illustrate such a process, which may be referred to as provider onboarding of patient 272.

A patient self-onboarding process 274 may proceed as depicted in FIG. 2C. The patient may create a system account profile through a web-based server website or by executing calls to the system API. The registration process may validate a phone number by sending a verification code. Once the registration account has been verified with the verification code, the user may be redirected to log in using his newly obtained credentials in the system identity server.

A two-step authentication process may be enforced for login as suggested by FIG. 6, either by authentication apps, or with a second factor of authentication through validated channels such as by phone or e-mail. Patient identity validation may also proceed as in FIG. 6. The system may request uploading of government issued photo identification, a photo of the user's face, and/or an insurance card. The system may optionally capture an image of a user's face and reconcile the image with the photo on the government issued identification for a match between the two images and flag users who do not closely match. The patient may alternatively or additionally provide information such as date of birth, street address, social security number for identity verification. A patient may optionally be verified against a database of known information and questions about address and financial history may be generated to prevent fraudulent patient right of accesses from being sent.

While logged in, the patient may sign the right of access form. The signed patient right of access form may then be stored in the system of record. Having verified right of access, the system may query 292, as in FIG. 2D, several claim resource providers that expose HIE APIs to retrieve claims and EOBs of patients. This may be done by querying systems that enforce the HL7 CARIN Alliance implementation for accessing real-time information through consumer-directed exchanges by following the below set of rules:
I—The message exchange and endpoint signatures of the claim network shall comply with HL7 FHIR standards for system interoperability.
II—To access the patient EOBs, the API retrieve methods shall conform to the HL7 FHIR CARINBB Implementation Guide STU1 v0.1.0 and support CARIN-BB-ExplanationOfBenefit (Base) and CARIN-BB-ExplanationOffienefit-Pharmacy profiles.
III—If not supported, similar profiles with structured data considered by the CARIN-BB Implementation guide shall be enforced for the system to access them. The claims and EOBs may be fetched using an HTTPS GET request under the FHIR standard, with a proper access token IDn by complying with the federation login scheme of each claim resource holder.

Provider and facility names returned 293 from the EOB and insurance claim data from the API may be aggregated. Patient identifying information and aggregated provider and facility names may be used in turn to query 295 the gateway 222 as can be seen in FIG. 2E. Virtual queries may be performed utilizing the IHE Technical Frameworks for Interoperability.

I—Providers may be queried to find patients using the IHE IT Infrastructure (ITI), Cross-Community Patient Discovery (XCPD) specification. This request to find patient profiles over providers follows the standard ITI-55 Initiating Gateway (ITI TF-2b: 3.55).
II—After the patient profile has been found, the provider may be queried over the list of current available health care records of the patient using ITI TF-1, Cross-Community Access (XCA) specification. This request to find patient documents over providers follows the standard ITI-38 Cross-Gateway Query for Initiating Gateway (ITI TF-2b: 3.38).
III—Once the proper documents have been found, the final request may be sent to retrieve the desired documents using ITI TF-1, Cross-Community Access (XCA) specification. This request to find patient documents over providers follows the standard ITI-39 Cross Gateway Retrieve for Initiating Gateway (ITI TF-2b: 3.39).

If no health insurance is presented, or the API comes back without any EOB, insurance claim, or other insurance-related results, or the patient wants to enter additional facilities or providers, then the patient may enter the names of their providers, and the system may use a central repository of provider names to send automated queries via fax 226, email 224, the gateway 222. The patient can additionally enter multiple providers to query the network. Providers may receive the data query via email 224, fax 226, or the gateway 222. The system may monitor requests that have no response within a set period of time, and flag such requests for an administrator to send a legal demand letter to the provider on the patient's behalf.

Providers who receive the query through the gateway 222 may respond using the same IHE Technical Frameworks for Interoperability as mentioned previously.

I—Providers respond to Cross-Community Patient Discovery (XCPD) requests using the standard ITI-55 Responding Gateway (ITI TF-2b: 3.55).
II—Cross-Community Access (XCA) queries for listing documents are answered using the standard ITI-38 Cross-Gateway Query for Responding Gateway (ITI TF-2b: 3.38).
III—Cross-Community Access (XCA) requests for retrieving documents are answered using the standard ITI-39 Cross Gateway Retrieve for Responding Gateway (ITT TF-2b: 3.39), applying MTOM transmission optimizations for attachments.

Providers who receive the right of access form through email 224 or fax 226 may be given a unique link to upload data for the patient making the request. Patient data received from the gateway 222 may be stored in the database automatically. Alternatively, provision of a unique link allows submission of patient health records through a secure web-based interface.

Providers can upload the medical data in any format (including PDF, HTML, JSON, FHIR, HL7, DICOM, JPEG, MP4, MOV, GIF, PNG, SNOMED CT, LOINC, RxNorm, XML, CDATA, TXT, TEXT, JPG, MP3, AVI, SVG, DOC, DOCX, XLS, XLSX, and raw binary) using the provider and payer portal 134. Patient data that was input into the database by the payers or providers may be assembled in the database.

Patient data may be segmented, as noted in FIG. 12A, using OCR and machine learning, into time series categorized for example as insurance claims, diagnosis, procedure, medication, provider, facility, laboratory values, allergies, immunizations, clinical images, and family history and normalized into FHIR. As can be seen from FIG. 12B, a data processing flow follows, according to four document types:

### a) Structured Data

i. Data in HL7 format, CDATA, XML, JSON, or other accepted format that has a unique interpretation and allows for direct import into the database.

### b) Known Format Files

i. Includes PDF, or similar files, that need to be recognized by OCR or similar techniques, for which the format of the entire document is known by the system and examples exist.
ii. The file is broken into sections by comparing images of the different sections, and then for each section OCR techniques are used to obtain the data.

### c) Known Document Category.

i. The type of study, record or information is known, but not the format.
ii. Hence, OCR techniques are used while searching for specific keywords under the domain of the file format.
iii. An overall description is produced, with a % of certainty, requiring review by the patient to validate the information.
iv. This information is used to complement the actual PDF or similar file.

### d) Unknown Type of Document

i. For these documents, the same approach is use as for the “Known document category”, with the difference that the information obtained is more generic and is only obtained for matters of searches and approximations. The original document is the only valid source of information, which gets wrapped around tags and information obtained by OCR.

Patients can combine multiple patient health records from multiple providers and payer. As in FIG. 13, the system may periodically re-query the provider and facility list to automatically update a PHR for new insurance or clinical events. The process may be repeated for different patients, and data for multiple patients may be aggregated and presented to the researcher in a graphical user interface (GUI). The patient can choose to authorize new providers, payors, or family to access their PHR and can revoke access. The patient can see a PHR sorted by time series categorized as, for example, diagnosis, insurance claims, procedure, medication, provider, facility, laboratory values, allergies, immunizations, clinical images, and family history.

Providers, payers and researchers have a visual representation of the patient in the database and can sort by time series categorized as, for example, diagnosis, insurance claims, procedure, medication, provider, facility, laboratory values, allergies, immunizations, clinical images, and family history. Providers, payers, and researchers can select a patient with specific insurance claims, diagnoses, procedures, medications, providers, facilities, laboratory values, allergies, immunizations, clinical images, and family histories of interest to aggregate into a patient pool. Providers, payers and researchers can subdivide patient pools and create a dashboard to monitor each variable on a time series basis. Providers, payers and researchers can use the graphical user interface to notify the patient pool of the payer's, provider's, or researcher's interest in the patient for communication and/or research. Providers, payers and researchers can consent to contact of the patient by the payers, providers, and/or researcher through the system. Patients can provide informed consent for contact in response to requests made by payers, providers, and/or researchers. After informed consent is received, payers, providers, and/or researchers can communicate freely with a patient using e-mail, text, and videoconferencing through the system.

PHR systems usually require a user to remember user login information for EMRs. An advantage of the claimed method and system is that a user does not need to remember login credentials for other systems, which is helpful for older populations.

The claimed system, from the patient's perspective, is automated and streamlined because a patient needs only to complete the registration process for records to begin being populated in the system of record.

An original copy of the PHR is kept, but the claimed methods and systems present a simplified view to the patient.

Patients can assemble records from multiple facilities and providers using the claimed methods and systems.

The patient right of access is a statutory requirement of a covered entity for a designated record set and carries a fine administered by the United States Department of Health and Human Services for non-conformance with valid patient right of access requests. The claimed methods and systems facilitate compliance with right of access requirements.

The claimed methods and systems allow researchers, providers and payers to access PHRs individually or in groups without concern for valid informed consent and permissions being properly given by the patient, which would be required if the PHI was not properly separated from the copy of the record provided to the researchers, providers, and payers.

The claimed methods and systems enable researchers to have access to anonymized data and patients who consent to contact for research purposes.

The claimed methods and systems allow payers, researchers, and patients to analyze health of individual patients and compare their health to a general population of patients having records on the system.

The claimed methods and systems reduce the complexity of retrieving a medical record and create a mechanism to automatically and cost effectively enforce compliance with the patient right of access.

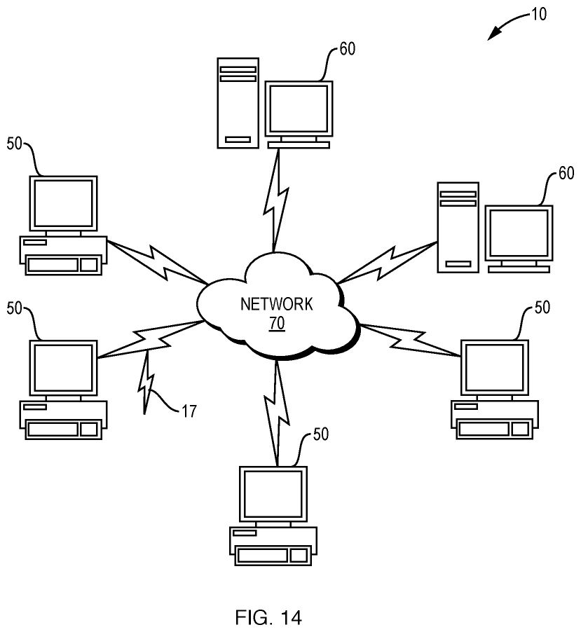

FIG. 14 illustrates a computer network (or system) 10 or similar digital processing environment, according to some embodiments of the present disclosure. Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, Near-Field Communication (NFC), etc.) to communicate with one another. Other electronic device/computer network architectures are suitable.

Client computers/devices 50 may be configured with a computing module (located at one or more of elements 50, 60, and/or 70). In some embodiments, a user may access the computing module executing on the server computers 60 from a user device, such a mobile device, a personal computer, or any computing device known to one skilled in the art without limitation. According to some embodiments, the client devices 50 and server computers 60 may be distributed across a computing module.

Server computers 60 may be configured as the computing modules which communicate with client devices 50 for providing access to (and/or accessing) databases that include patient healthcare data. The server computers 60 may not be separate server computers but part of cloud network 70. In some embodiments, the server computer (e.g., computing module) may enable management of access to patient healthcare data by allowing access to data located on the client 50, server 60, or network 70 (e.g., global computer network). The client (configuration module) 50 may communicate data representing the patient healthcare data back to and/or from the server (computing module) 60. In some embodiments, the client 50 may include client applications or components executing on the client 50 for managing access to patient healthcare data, and the client 50 may communicate corresponding data to the server (e.g., computing module) 60.

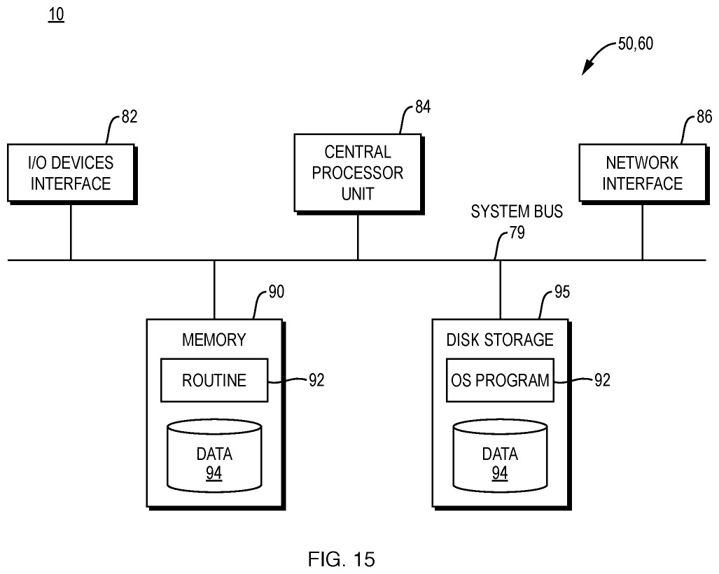

Some embodiments of the system 10 may include a computer system for managing access to patient healthcare data. The system 10 may include a plurality of processors 84. The system 10 may also include a memory 90. The memory 90 may include: (i) computer code instructions stored thereon; and/or (ii) data representing patient insurance or medical history data. The data may include segments including portions of the patient insurance or medical history data. The memory 90 may be operatively coupled to the plurality of processors 84 such that, when executed by the plurality of processors 84, the computer code instructions may cause the computer system 10 to implement a computing module (the computing module being located on, in, or implemented by any of elements 50, 60, 70 of FIG. 14 or elements 82, 84, 86, 90, 92, 94, 95 of FIG. 15) configured to perform one or more functions.

According to some embodiments, FIG. 15 is a diagram of an example internal structure of a computer (e.g., client processor/device 50 or server computers 60) in the computer system 10 of FIG. 15. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 14). Memory 90 provides volatile storage for computer software instructions 92 and data 94 used to implement some embodiments (e.g., patient healthcare data described herein). Disk storage 95 provides non-volatile storage for computer software instructions 92 and data 94 used to implement an embodiment of the present disclosure. A central processor unit 84 is also attached to the system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the present disclosure. The computer program product 92 can be installed by any suitable software installation procedure, as is well known in the art. In another embodiment, at least a portion of the software instructions may also be downloaded over a cable, communication and/or wireless connection. Other embodiments may include a computer program propagated signal product 107 (of FIG. 14) embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the routines/program 92 of the present disclosure.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

Embodiments or aspects thereof may be implemented in the form of hardware (including but not limited to hardware circuitry), firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

While example embodiments have been particularly shown and described, it will be understood by those skilled in the art that various changes in form and details may be made therein without departing from the scope of the embodiments encompassed by the appended claims.

What is claimed is:

1. A processor-implemented method of managing access to patient healthcare data comprising:
registering a patient for access to a health information exchange (HIE) system;
providing login credentials to the patient;
granting the patient access to the HIE system on an identity server by validating login credentials submitted by the patient against the provided login credentials;
obtaining, from the patient, information pertaining to the patient including an identity of the patient;
validating the received identity of the patient;
generating a right of access form and providing the right of access form to the patient;
obtaining an acceptance of a right of access form from the patient;
storing the right of access form in a memory device of at least one of the identity server and a user health database server;
receiving a query for healthcare data pertaining to the patient from a querying entity;
relaying a query for healthcare data toward a possessing entity;
obtaining the healthcare data, or an indication of an error, from the possessing entity through the HIE system;
relaying the obtained healthcare data, or the indication of an error, to the querying entity; and
creating or augmenting a personal health record (PHR) by storing the obtained healthcare data in a memory device of at least one of the identity server and the user health database server.

2. The method of claim 1 wherein registering a patient includes:
establishing, on the identity server, an account profile for the patient;
populating the account profile with initial profile information of the patient obtained by at least one of (i) receiving the initial profile information through a web-based server website or (ii) by executing calls for the initial profile information to an application programming interface (API);
providing a verification code to the patient to verify a piece of contact information of the initial profile information, receiving a return verification code from the patient, and completing registering the patient for access if a match between the provided and received verification codes is detected upon comparison of the provided and received verification codes.

3. The method of claim 1 wherein the login credentials include at least one of a validation code, a username, a password, and a successful completion of a second-factor authentication process.

4. The method of claim 1 wherein the information pertaining to the patient includes at least one of identity documents or information therefrom, an insurance card or information therefrom, and a name of a medical provider.

5. The method of claim 4 wherein the medical provider is a primary care physician (PCP) or a specialist.

6. The method of claim 1 wherein validating the received identity of the patient includes accepting at least one of a government issued photographic identification, a photograph of the patient's face, and an insurance card.

7. The method of claim 6 further comprising capturing an image of the patient's face and reconciling the captured image of the patient's face with the accepted government issued photographic identification or the accepted photograph of the patient's face.

8. The method of claim 1 wherein obtaining the healthcare data includes:
accessing a record locator service (RLS) database;
searching the RLS database, based on proximity to an address represented by address information pertaining to the patient, for providers associated with the patient; and
identifying at least one provider, based on the searching, from which to obtain healthcare data pertaining to the patient.

9. The method of claim 1 wherein the healthcare data pertaining to the patient includes at least one of past insurance claim data or a name of a medical provider obtained therefrom, and past data from an explanation of benefits (EOB) form or a name of a medical provider obtained therefrom, and the obtained healthcare data is stored on at least one of the identity server, the user health database server, and combinations thereof.

10. The method of claim 1 wherein the healthcare data pertaining to the patient includes medical data of a health record of the patient.

11. The method of claim 1 wherein the querying entity is at least one of the patient, a medical provider, and a medical researcher.

12. The method of claim 1 wherein a telemedicine session is established between the patient and the querying entity, further comprising confirming possession of the right of access form executed by the patient and stored in a memory device of at least one of the identity server and the user health database server, and further comprising obtaining informed consent from the patient prior to establishing the telemedicine session.

13. The method of claim 1 wherein the querying entity is a medical researcher, further comprising:
confirming possession of the right of access form executed by the patient and stored in the memory device;
obtaining informed consent from the patient;
anonymizing the obtained healthcare data of the patient and including the anonymized healthcare data of the patient in a pool of healthcare data of multiple patients.

14. The method of claim 1 wherein the querying entity is the HIE system, and the HIE system is configured to automatically self-receive the query and to automatically relay the query toward the possessing entity.

15. The method of claim 14 wherein the HIE system is configured to automatically update the PHR by periodically querying possessing entities on behalf of the patient.

16. The method of claim 1 wherein the possessing entity is an insurance provider or a medical provider.

17. The method of claim 1 wherein the possessing entity is a server of the HIE, further comprising relaying the query from the querying entity to the possessing entity, or to at least one of the HIE, the API, and a gateway of the HIE, the server being at least one of the identity server and the user health database server.

18. The method of claim 1 wherein the query for healthcare data is relayed from the querying entity toward the possessing entity via a gateway of the HIE, or via e-mail or fax.

19. The method of claim 1 wherein the healthcare data, or the indication of an error, is obtained by providing the possessing entity with a secure unique hyperlink or uniform resource locator (URL) leading to a provider and payer portal of the HIE system.

20. The method of claim 1 wherein the obtained healthcare data is received in a format of any of PDF, HTML, JSON, FHIR, HL7, DICOM, JPEG, MP4, MOV, GIF, PNG, SNOMED CT, LOINC, RxNorm, XML, CDATA, TXT, TEXT, JPG, MP3, AVI, SVG, DOC, DOCX, XLS, XLSX, and raw binary.

21. The method of claim 1 further including segmenting, using machine learning, the obtained healthcare data into time series categorized at least as insurance claims, diagnosis, medication, procedure, provider, facility, laboratory values, allergies, immunizations, clinical images, and family history.

22. The method of claim 21 wherein segmenting the obtained healthcare data includes:
classifying the obtained healthcare data as structured data or unstructured data;
classifying unstructured data as being of a known format or of an unknown format;
classifying unstructured data of an unknown format as being of a document or study of a known type;
prior to storing the obtained healthcare data, wrapping in a shell document the obtained healthcare data classified as unstructured data of unknown format of unknown document or study type, the document having a CDATA format;
converting unstructured data of unknown format of known document or study type to structured data by performing optical character recognition (OCR) based on pattern recognition for known terms of study;
converting unstructured data of known format to structured data by performing OCR based on pattern recognition for terms presently stored on user health database server; and
prior to storing the obtained healthcare data, converting, to a standard format, the obtained healthcare data classified as or converted to structured data.

23. The method of claim 22 wherein the standard format is defined by a Fast Healthcare Interoperability Resources (FHIR) specification.

24. The method of claim 21 further including:
determining, based on at least one of the categorized time series, a health score for the patient;
classifying a health condition of the patient based on the health score, the health condition including an assessment of a likelihood of a patient to be dangerous to public health; and
issuing a certificate that attests to information of a patient including at least one of a PHR history, a health score, and a health condition, the certificate being a printable document including a URL, or a representation thereof, leading to a webpage enabling retrieval from the HIE of a copy of the certificate, or a representation thereof, to demonstrate authenticity of the certificate.

25. The method of claim 1 further including, subsequent to relaying the query for healthcare data to a possessing entity, determining that a specified period of time has passed prior to obtaining the healthcare data from the possessing entity, and prompting an administrator or a legal entity to send a legal demand letter regarding the healthcare data to the possessing entity on behalf of the patient.

26. A system for managing access to patient healthcare data comprising a processor and a memory device with instructions loaded thereon, the instructions, when loaded, configuring the processor to:
register a patient for access to a health information exchange (HIE) system;
provide login credentials to the patient;
grant the patient access to the HIE system on an identity server by validating login credentials submitted by the patient against the provided login credentials;
obtain, from the patient, information pertaining to the patient including an identity of the patient;
validate the received identity of the patient;
generate a right of access form and provide the right of access form to the patient;
obtain an executed right of access form from the patient;
store the right of access form in a memory device of at least one of the identity server and a user health database server;
receive a query for healthcare data pertaining to the patient from a querying entity;
relay a query for healthcare data toward a possessing entity;
obtain the healthcare data, or an indication of an error, from the possessing entity through the HIE system;
relay the obtained healthcare data, or the indication of an error, to the querying entity; and
create or augment a personal health record (PHR) by storing the obtained healthcare data in a memory device of at least one of the identity server and the user health database server.

27. The system of claim 26 wherein the instructions to configure the processor to register a patient include instructions to configure the processor to:
establish, on the identity server, an account profile for the patient;
populate the account profile with initial profile information of the patient obtained by at least one of (i) receiving the initial profile information through a web-based server website or (ii) by executing calls for the initial profile information to an application programming interface (API); and
provide a verification code to the patient to verify a piece of contact information of the initial profile information, receiving a return verification code from the patient, and completing registering the patient for access if a match between the provided and received verification codes is detected upon comparison of the provided and received verification codes.

28. The system of claim 26 wherein the login credentials include at least one of a validation code, a username, a password, and a successful completion of a second-factor authentication process.

29. The system of claim 26 wherein the information pertaining to the patient includes at least one of identity documents or information therefrom, an insurance card or information therefrom, and a name of a medical provider.

30. The system of claim 26 wherein the medical provider is a primary care physician (PCP) or a specialist.

31. The system of claim 26 wherein the instructions to configure the processor to validate the received identity of the patient include instructions to configure the processor to accept of at least one of a government issued photographic identification, a photograph of the patient's face, and an insurance card.

32. The system of claim 31 further comprising an image capture device configured to capture an image of the patient's face, and wherein the processor is further configured to reconcile the captured image of the patient's face with the accepted government issued photographic identification or the accepted photograph of the patient's face.

33. The system of claim 26 wherein the instructions to configure the processor to obtain the healthcare data include instructions to configure the processor to:
access a record locator service (RLS) database;
search the RLS database, based on proximity to an address represented by address information pertaining to the patient, for providers associated with the patient; and
identify at least one provider, based on the searching, from which to obtain healthcare data pertaining to the patient.

34. The system of claim 26 wherein the healthcare data pertaining to the patient includes at least one of past insurance claim data or a name of a medical provider obtained therefrom, and past data from an explanation of benefits (EOB) form or a name of a medical provider obtained therefrom, and the processor is configured to store the obtained healthcare data on at least one of the identity server, the user health database server, and combinations thereof.

35. The system of claim 26 wherein the healthcare data pertaining to the patient includes medical data of a health record of the patient.

36. The system of claim 26 wherein the querying entity is at least one of the patient, a medical provider, and a medical researcher.

37. The system of claim 26 wherein a telemedicine session is established between the patient and the querying entity, and the processor is further configured to confirm possession of the right of access form executed by the patient and stored in a memory device of at least one of the identity server and the user health database server, and further comprising obtaining informed consent from the patient prior to establishing the telemedicine session.

38. The system of claim 26 wherein the querying entity is a medical researcher and the processor is further configured to:
confirm possession of the right of access form executed by the patient and stored in the memory device;
obtain informed consent from the patient;
anonymize the obtained healthcare data of the patient and include the anonymized healthcare data of the patient in a pool of healthcare data of multiple patients.

39. The system of claim 26 wherein the querying entity is the HIE system, and the HIE system is configured to automatically self-receive the query and to automatically relay the query toward the possessing entity.

40. The system of claim 39 wherein the HIE system is configured to automatically update the PHR by periodically querying possessing entities on behalf of the patient.

41. The system of claim 26 wherein the possessing entity is an insurance provider or a medical provider.

42. The system of claim 26 wherein the possessing entity is a server of the HIE, and the processor is further configured to relay the query from the querying entity to the possessing entity or to at least one of the HIE, the API, and a gateway of the HIE, the server being at least one of the identity server and the user health database server.

43. The system of claim 26 wherein the processor is configured to relay the query for healthcare data from the querying entity toward the possessing entity via a gateway of the HIE, or via e-mail or fax.

44. The system of claim 26 wherein the processor is configured to obtain the healthcare data, or the indication of an error, by providing the possessing entity with a secure unique hyperlink or uniform resource locator (URL) leading to a provider and payer portal of the HIE system.

45. The system of claim 26 wherein the processor is configured to receive the obtained healthcare data in a format of any of PDF, HTML, JSON, FHIR, HL7, DICOM, JPEG, MP4, MOV, GIF, PNG, SNOMED CT, LOINC, RxNorm, XML, CDATA, TXT, TEXT, JPG, MP3, AVI, SVG, DOC, DOCX, XLS, XLSX, and raw binary.

46. The system of claim 26 wherein the processor is further configured to segment, using machine learning, the obtained healthcare data into time series categorized at least as insurance claims, diagnosis, medication, procedure, provider, facility, laboratory values, allergies, immunizations, clinical images, and family history.

47. The system of claim 46 wherein the instructions to configure a processor to segment the obtained healthcare data include instructions to configure the processor to:
classify the obtained healthcare data as structured data or unstructured data;
classify unstructured data as being of a known format or of an unknown format;
classify unstructured data of an unknown format as being of a document or study of a known type;
prior to storing the obtained healthcare data, wrap in a shell document the obtained healthcare data classified as unstructured data of unknown format of unknown document or study type, the document having a CDATA format;
convert unstructured data of unknown format of known document or study type to structured data by performing optical character recognition (OCR) based on pattern recognition for known terms of study;
convert unstructured data of known format to structured data by performing OCR based on pattern recognition for terms presently stored on user health database server; and
prior to storing the obtained healthcare data, convert, to a standard format, the obtained healthcare data classified as or converted to structured data.

48. The system of claim 47 wherein the standard format is defined by a Fast Healthcare Interoperability Resources (FHIR) specification.

49. The system of claim 46 wherein the processor is further configured to:
determine, based on at least one of the categorized time series, a health score for the patient;
classify a health condition of the patient based on the health score, the health condition including an assessment of a likelihood of a patient to be dangerous to public health; and
issue a certificate that attests to information of a patient including at least one of a PHR history, a health score, and a health condition, the certificate being a printable document including a URL, or a representation thereof, leading to a webpage enabling retrieval from the HIE of a copy of the certificate, or a representation thereof, to demonstrate authenticity of the certificate.

50. The system of claim 26 wherein the processor is further configured, subsequent to relaying the query for healthcare data to a possessing entity, to determine that a specified period of time has passed prior to obtaining the healthcare data from the possessing entity, and prompt an administrator or a legal entity to send a legal demand letter regarding the healthcare data to the possessing entity on behalf of the patient.
